(19) United States
(12) Patent Application Publication
(10) Pub. No.: US 2005/0120494 A1
(43) Pub. Date: Jun. 9, 2005

(54) Triazacyclononane derivatives substituted on at least one of the nitrogen atoms with a substituted or unsubstituted 4'-aminophenyl group, for dyeing keratin fibres

(76) Inventors: Laurent Vidal, Paris (FR); Stephane Sabelle, Paris (FR); Thi-My Ly-Carry, St. Maur (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/948,793

(22) Filed: Sep. 24, 2004

(30) Foreign Application Priority Data
Sep. 26, 2003 (FR) 03 11307

(51) Int. Cl. A61K 7/13

(52) U.S. Cl. 8/405; 540/474

(57) ABSTRACT

The invention relates to triazacyclononane derivatives substituted on at least one of the nitrogen atoms with a substituted or unsubstituted 4′-aminophenyl group, and also to the use thereof as oxidation bases for dyeing keratin fibres and in particular human keratin fibres such as the hair. The invention also relates to a composition for dyeing keratin fibres, and in particular human keratin fibres such as the hair, comprising at least one triazacyclononane derivative substituted on at least one of the nitrogen atoms with a 4′-aminophenyl group, as oxidation base, and also to the dyeing process using this composition. The present invention makes it possible in particular to obtain strong, sparingly selective and fast coloration of keratin fibres.

The invention relates to triazacyclononane derivatives substituted on at least one of the nitrogen atoms with a substituted or unsubstituted 4′-aminophenyl group, and also to the use thereof as oxidation bases for dyeing keratin fibres and in particular human keratin fibres such as the hair.

It is known practice to dye keratin fibres, and in particular human keratin fibres such as the hair, with dye compositions containing oxidation dye precursors, in particular ortho- or para-phenylenediamines, ortho- or para-aminophenols, heterocyclic compounds such as diaminopyrazole derivatives, pyrazolo[1,5-a]pyrimidine derivatives, pyrimidine derivatives, pyridine derivatives, 5,6-dihydroxyindole derivatives and 5,6-dihydroxyindoline derivatives, which are generally known as oxidation bases. Oxidation dye precursors, or oxidation bases, are colourless or weakly coloured compounds that, when combined with oxidizing products, may give rise to coloured compounds or dyes by a process of oxidative condensation.

It is also known that the shades obtained with these oxidation bases may be varied by combining them with couplers or coloration modifiers, the latter being chosen especially from meta-phenylenediamines, meta-aminophenols, meta-hydroxyphenols and certain heterocyclic compounds, for instance pyrazolo[1,5-b]-1,2,4-triazole derivatives, pyrazolo[3,2-c]-1,2,4-triazole derivatives, pyrazolo[1,5-a]pyrimidine derivatives, pyridine derivatives, pyrazol-5-one derivatives, indoline derivatives and indole derivatives.

The variety of molecules used as oxidation bases and couplers allows a wide range of colours to be obtained.

The “permanent” coloration obtained using these oxidation dyes must moreover satisfy a certain number of requirements. Thus, it must have no toxicological drawback, it must be able to produce shades in the desired intensity, and it must show good resistance to external agents such as light, bad weather, washing, permanent-waving, perspiration and rubbing.

The dyes must also be able to cover white hairs and, finally, they must be as unselective as possible, i.e. they must produce the smallest possible differences in coloration along the same keratin fibre, which may in fact be differently sensitized (i.e. damaged) between its end and its root. They must also show good chemical stability in the formulations and must have a good toxicological profile.

In the field of hair dyeing, para-phenylene-diamine and para-tolylenediamine are oxidation bases that are widely used. They make it possible with oxidation couplers to obtain varied shades.

However, there is a need to discover new oxidation bases that have a better toxicological profile than para-phenylenediamine and para-tolylene-diamine, while at the same time giving the hair excellent properties in terms of colour intensity, variety of shade, colour uniformity and resistance to external agents.

It is already known practice to use 1,4-diazacycloheptane derivatives for the oxidation dyeing of keratin fibres. For example, patent U.S. Pat. No. 6,165,230 describes a dye composition comprising 1,4-diaza-cycloheptane derivatives substituted on the two nitrogen atoms of the ring with a 4′-aminophenyl group.

It is also known practice to use 1,4,7-triazacyclononane derivatives to dye keratin fibres. For example, patent JP 2002-255763 describes a dye composition comprising 1,4,7-triazacyclononane derivatives substituted on one or more nitrogen atoms of the ring with a 2′-aminobenzyl group.

It is clearly established that these compounds cannot give the hair a coloration equivalent in quality to that obtained with para-phenylenediamine or with para-tolylenediamine, due to the lack of colour intensity and colour uniformity.

There is thus a real need to discover novel oxidation bases that have both a good toxicological profile and properties such that the dye compositions containing them can give the hair excellent properties in terms of colour intensity, variety of shades, colour uniformity and resistance to the various external attacks to which the hair may be subjected.

The aim of the present invention is to develop novel oxidation bases for dyeing keratin fibres, which do not have the drawbacks of the oxidation bases of the prior art, by providing novel compounds for dyeing keratin fibres that do not degrade the keratin fibres, while at the same time being capable of producing strong colorations in varied shades, which are sparingly selective and particularly resistant, and which have a good toxicological profile.

This aim is achieved with the present invention, one subject of which is triazacyclononane derivatives substituted on at least one of the nitrogen atoms with a substituted or unsubstituted 4′-aminophenyl group.

The present invention makes it possible in particular to obtain strong, sparingly selective and fast dyeing of keratin fibres.

Another subject of the invention is the use of these triazacyclononane derivatives as oxidation bases for dyeing keratin fibres, and in particular human keratin fibres such as the hair.

A subject of the invention is also a composition for dyeing keratin fibres, and in particular human keratin fibres such as the hair, comprising at least one triazacyclononane derivative as defined above as oxidation base, and also the process for dyeing keratin fibres using this composition.

Finally, a subject of the invention is triazacyclononane derivatives substituted on at least one of the nitrogen atoms with a substituted or unsubstituted 4′-nitrophenyl group, for synthesizing the triazacyclononane derivatives as defined above.

According to one particular embodiment of the invention, the triazacyclononane derivatives substituted on at least one of the nitrogen atoms with a substituted or unsubstituted 4′-aminophenyl group are chosen from the compounds of formula (I) below, and the addition salts thereof: 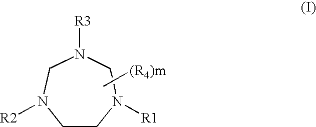
in which:
R₁, R₂ and R₃ represent:
   a hydrogen atom;
   a saturated or unsaturated alkyl radical, which is unsubstituted or substituted with one or more carboxyl, alkylcarbonyl, alkoxycarbonyl, carbamoyl, mono- or dialkylcarbamoyl, alkylsulfonyl, trialkylammonium or N-alkylimidazolium radicals, or saturated or unsaturated heterocyclic radicals containing 4, 5, 6 or 7 atoms, the hetero atom(s) of which is (are) chosen from nitrogen, oxygen and sulfur;
   a radical —CH₂—R′ in which R′ is a saturated or unsaturated alkyl radical substituted with one or more hydroxyl, alkoxy, amino, mono- or dialkylamino, trialkylammonium, alkylcarbonylamino or alkylcarbonyloxy radicals;
   a saturated or unsaturated alkylcarbonyl radical, which is unsubstituted or substituted with one or more hydroxyl, alkoxy, amino, mono- or dialkylamino, alkylsulfonyl, carboxyl, alkylcarbonyl, alkoxycarbonyl, carbamoyl, mono- or dialkylcarbamoyl, trialkylammonium or N-alkylimidazolium radicals, or saturated or unsaturated heterocyclic radicals containing 4, 5, 6 or 7 atoms, the hetero atom(s) of which is (are) chosen from nitrogen, oxygen and sulfur;
   an alkoxycarbonyl radical;
   a mono- or dialkylcarbamoyl radical;
   a carbamoyl radical;
   a carboxyl radical;
   an alkylsulfonyl radical;
   an arylsulfonyl radical;
   a radical corresponding to formula (II): 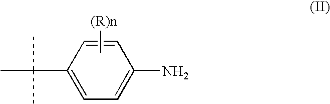
      in which:
R represents:
   a saturated or unsaturated alkyl radical, which is unsubstituted or substituted with one or more hydroxyl, amino, guanidinyl, trialkylammonium, N-alkylimidazolium, carboxyl or carbamoyl radicals;
   a saturated or unsaturated alkoxy radical, which is unsubstituted or substituted with one or more hydroxyl, amino, trialkylammonium or N-alkylimidazolium radicals;
   an alkoxyalkyl radical;
   a hydroxyalkoxyalkyl radical;
   an aminoalkyl radical, the amine possibly being unsubstituted or mono- or disubstituted with an alkyl, acetyl or hydroxyalkyl radical;
n is between 0 and 4, it being understood that when n is greater than 1, then the radicals R may be identical or different;
   it being understood that at least one of the radicals R₁, R₂ and R₃ is chosen from the radicals of formula (II);
R₄ represents:
   a saturated or unsaturated alkyl radical;
   a hydroxyalkyl radical;
   an alkoxyalkyl radical;
   an alkylcarbonyl radical;
   a hydroxyalkoxyalkyl radical;
   an aminoalkyl radical, the amine possibly being unsubstituted or mono- or disubstituted with an alkyl, acetyl or hydroxyalkyl radical;
   a hydroxyl or aminoalkyl radical;
   a carboxyl radical;
   a carboxyalkyl radical;
   a carbamoyl radical;
   a carbamoylalkyl radical;
   an alkoxycarbonyl radical;
   a mono- or dialkylaminocarbonyl radical;
m is between 0 and 6, it being understood that when m is greater than 1, then the radicals R₄ may be identical or different.

In the above definitions, the alkyl radicals are linear or branched and comprise, unless otherwise indicated, from 1 to 10 carbon atoms and preferably from 1 to 6 carbon atoms. An alkoxy radical is an alkyl-O— radical, the alkyl radical being as defined above.

The term “unsaturated alkyl radical” means an alkyl radical of 2 to 10 carbon atoms comprising one or more double and/or triple bonds.

A substituted alkyl radical is a mono- or polysubstituted alkyl radical. For example, a hydroxyalkyl radical or an aminoalkyl radical is an alkyl radical that may be substituted with one or more hydroxyl or amino groups.

When the derivatives of formula (I) are cationic, for example when one of the radicals R₁, R₂ and R₃ represents an alkyl radical substituted with a trialkylammonium radical, they are associated with one or more counterions chosen from mineral anions such as halides, hydroxides, sulfates and hydrogen sulfates, or organic anions such as acetates, citrates, tartrates, alkyl sulfates in which the linear or branched alkyl portion is of C₁-C₆, alkyl sulfonates for which the linear or branched alkyl portion is of C₁-C₆, and aryl sulfonates for which the aryl portion, preferably phenyl, is optionally substituted with one or more C₁-C₄ alkyl radicals.

In the derivatives of formula (I), the radical R is chosen, for example, from a methyl radical; an ethyl radical; an isopropyl radical; a vinyl radical; an allyl radical; a methoxymethyl radical; a hydroxymethyl radical; a 1-carboxymethyl radical; a 1-aminomethyl radical; a 2-carboxyethyl radical; a 2-hydroxyethyl radical; a 3-hydroxypropyl radical; a 1,2-dihydroxyethyl radical; a 1-hydroxy-2-aminoethyl radical; a 1-amino-2-hydroxyethyl radical; a 1,2-diaminoethyl radical; a methoxy radical; an ethoxy radical; an allyloxy radical; a 2-hydroxyethyloxy radical.

According to one particular embodiment of the invention, two of the radicals R₁, R₂ and R₃ are radicals corresponding to formula (II).

According to one particular embodiment of the invention, n is equal to 0 or R is chosen from an alkyl radical; a hydroxyalkyl radical; an aminoalkyl radical; an alkoxy radical; a hydroxyalkoxy radical. By way of example, R may be a methyl radical; a hydroxymethyl radical; a 1,2-dihydroxyethyl radical.

According to one preferred embodiment of the invention, n is equal to 0 or 1.

According to one particular embodiment of the invention, the radical(s) R₁, R₂ and/or R₃, which are different from the radicals corresponding to formula (II), are chosen from a hydrogen atom; an alkyl radical; an alkylcarbonyl radical; an alkylsulfonyl radical; a radical —CH₂—R′ in which R′ is an alkyl radical substituted with one or more hydroxyl, alkoxy, amino, mono- or dialkylamino or trialkylammonium radicals; an alkyl radical substituted with one or more trialkylammonium or N-alkylimidazolium radicals; an alkylcarbonyl radical substituted with one or more N-alkylimidazolium radicals; a mono- or dialkylcarbamoyl radical. By way of example, the radical(s) R₁, R₂ and/or R₃, which are different from the radicals corresponding to formula (II), may be a hydrogen atom; a 2hydroxyethyl radical; a 2,3-dihydroxypropyl radical; a 3-hydroxypropyl radical; a 2-aminoethyl radical; a 2-methoxyethyl radical; a methyl radical; an ethyl radical; an acetyl radical; a methylsulfonyl radical; an N,N-dimethylcarbamoyl radical; a 2-(N,N-dimethylamino)ethyl radical; a (1-methylimidazol-1-ium-3-yl)methylcarbonyl radical; a 3-(trimethylammonium)-2-hydroxypropyl radical; a (1-methylimidazol-1-ium-3-yl)propyl radical; a 2-(trimethylammonium)ethyl radical.

According to one particular embodiment of the invention, m is equal to 0 or R₄ is chosen from an alkyl radical; a hydroxyalkyl radical; a carboxyl radical; a carbamoyl radical.

According to one preferred embodiment of the invention, m is equal to 0 or 1.

The triazacyclononane compounds as defined above may be optionally salified with strong mineral acids, for instance HCl, HBr or H₂SO₄, or organic acids, for instance acetic acid, lactic acid, tartaric acid, citric acid or succinic acid.

As examples of triazacyclononane derivatives in accordance with the invention, mention may be made of the compounds presented in the table below.

| Formula | Nomenclature |
|---|---|
| 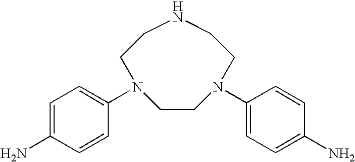 | 1,4-bis(4-aminophen- yl)[1,4,7]triazonane |
| 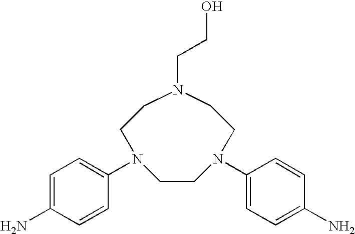 | 2-[4,7-bis(4-amino- phenyl)[1,4,7]tria- zonan-1-yl]ethanol |
| 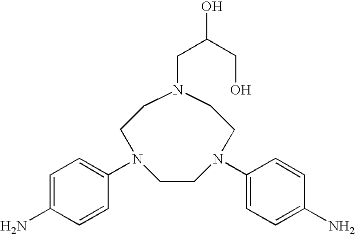 | 3-[4,7-bis(4-amino- phenyl)[1,4,7]tri- azonan-1-yl]propane- 1,2-diol |
| 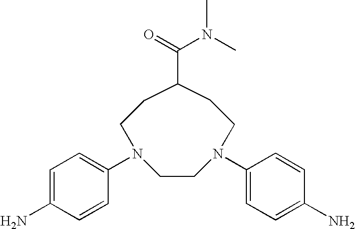 | 1-(N,N-dimethylcar- bamoyl)-4,7-bis(4- aminophenyl)[1,4,7]- triazonaane |
| 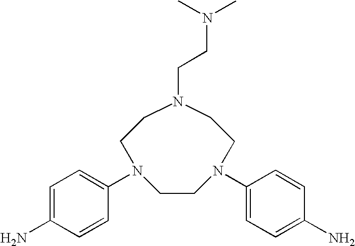 | 1-[β-(N,N-dimethyl- amino)ethyl]-4,7- bis(4-aminophenyl)- [1,4,7]triazonane |
| 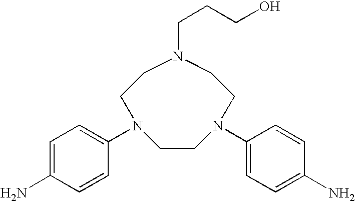 | 1-(γ-hydroxypropyl)_- 4,7-bis(4-aminoiphen- yl)[1,4,7]triazonane |
| 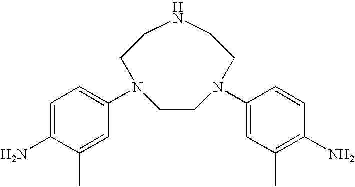 | 1,4-bis(4-amino-3- methylphenyl)[1,4,7]- triazonane |
| 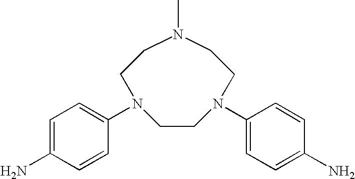 | 1-methyl-4,7-bis(4- aminophenytl)[1,4,7]- triazonane |
| 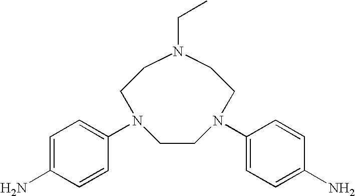 | 1-ethyl-4,7-bis(4- aminophenyl)[1,4,7]- triazonane |
| 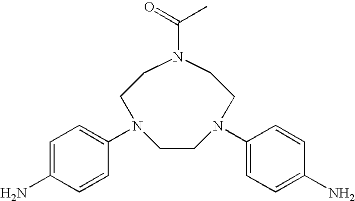 | 1-acetyl-4,7-bis(4- aminophenyl)[1,4,7]- triazonane |
| 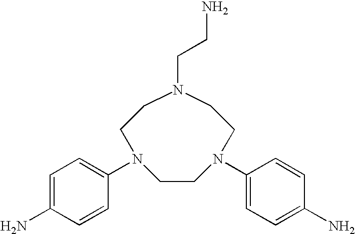 | 1-(β-aminoethyl)-4,7- bis(4-aminophen- yl)[1,4,7]triazonane |
| 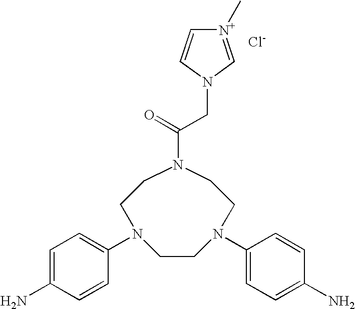 | 3-{2-[4,7-bis(4-ami- nophenyl)[1,4,7]tri- azonan-1-yl]-2-oxo- ethyl}-1-methyl-3H- imidazol-1-ium chloride |
| 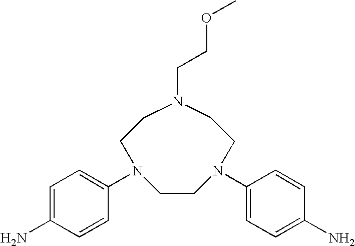 | 1-(β-methoxyethyl)- 4,7-bis(4-aminophen- yl)[1,4,7]triazonane |
| 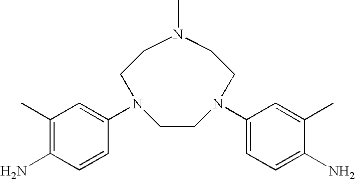 | 1-methyl-4,7-bis(4- amino-3-meethylphen- yl)[1,4,7]triazonane |
| 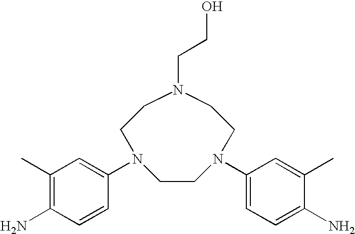 | 2-[4,7-bis(4-amino-3- methylphenyl)[1,4,7]- triazonan-1-yl]eth- anol |
| 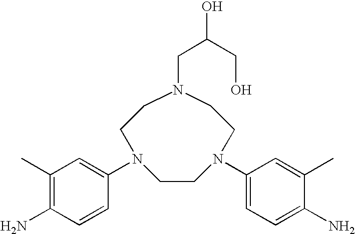 | 3-[4,7-bis(4-amino-3- methylphenyl)[1,4,7]- triazonan-1-yl]pro- pane-1,2-diol |
| 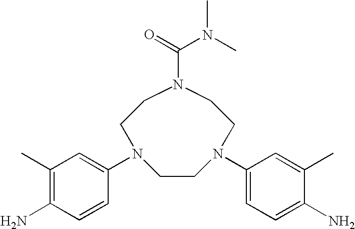 | 1-(N,N-dimethylcar- bamoyl)-4,7-bis(4- amino-3-methylphen- yl)[1,4,7]triazonane |
| 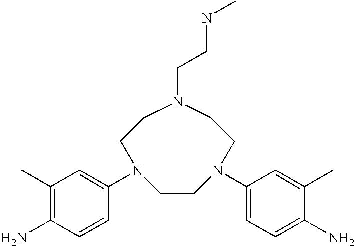 | 1-[β-(N,N-dimethylam- ino)ethyl]-4,7-bis(4- amino-3-methylphen- yl)[1,4,7]triazonane |
| 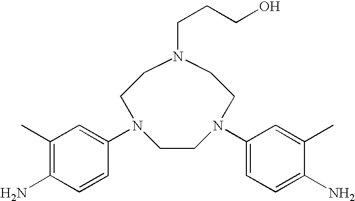 | 3-[4,7-bis(4-amino-3- methylphenyl)[1,4,7]- triazonan-1- yl]propan-1-ol |
| 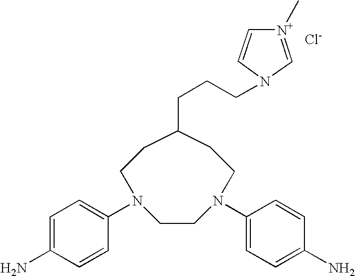 | 3-{3-[4,7-bis(4-ami- nophenyl)[1,4,7]tri- azonan-1-yl]propyl}- 1-methyl-3H-imidazol- 1-ium chloride |
| 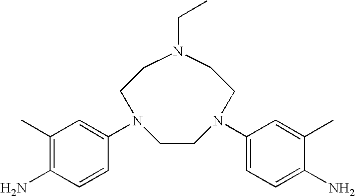 | 1-ethyl-4,7-bis(4- amino-3-methylphen- yl)[1,4,7]triazonane |
| 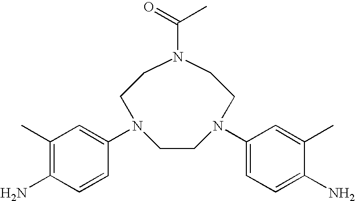 | 1-acetyl-4,7-bis(4- amino-3-methylphen- yl)[1,4,7]triazonane |
| 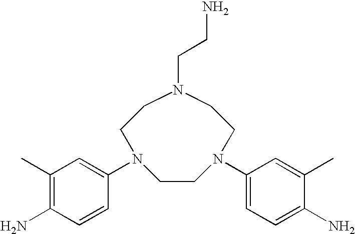 | 1-(β-aminoethyl)-4,7- bis(4-amino-3- methylphenyl)[1,4,7]- triazonane |
| 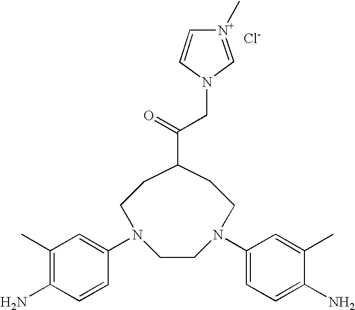 | 3-{2-[4,7-bis(4-am- ino-3-methylphenyl)- [1,4,7]triazonan-1- yl]-2-ocxoethyl}-1- methyl-3H-imidazol- 1-ium chloride |
| 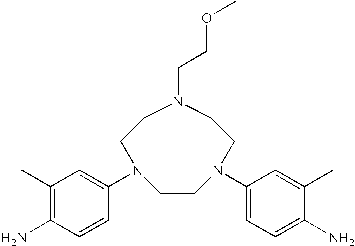 | 1-(β-methoxyethyl)- 4,7-bis(4-amino-3- methylphenyl)[1,4,7]- triazonane |
| 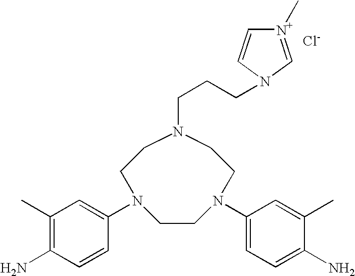 | 3-{3-[4,7-bis(4-ami- no-3-methylphenyl)- [1,4,7]triazonan-1- yl]propyl}-1-methyl- 3H-imidazol-1-ium |
| 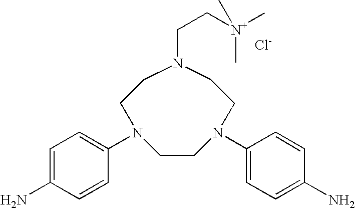 | (2-[4,7-bis(4-amino- phenyl)[1,4,7]tri- azonan-1-yl]ethyl}- trimethylammonium chloride |
| 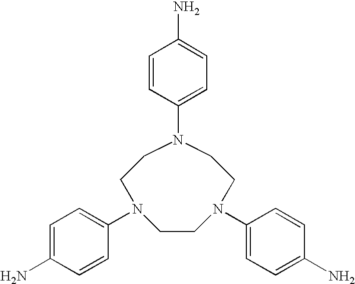 | 1,4,7-tris(4-amino- phenyl)[1,4,7]triazo- nane |
| 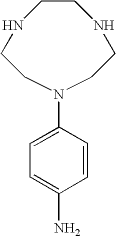 | 1-(4-aminophenyl)- [1,4,7]triazonane |
| 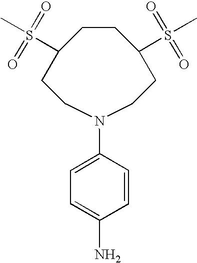 | 1,4-bis(methanesulf- onyl)-7-(4-aminophen- yl)[1,4,7]triazonane |
| 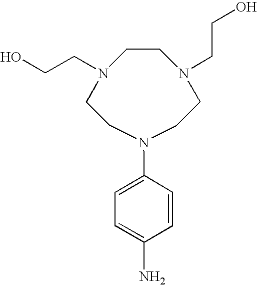 | 1,4-bis(β-hydroxy- ethyl)-7-(4-amino- phenyl)[1,4,7]tri- azonane |
| 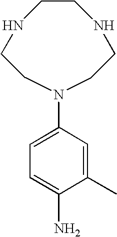 | 1-(4-amino-3-methyl- phenyl)[1,4,7]tri- azonane |
| 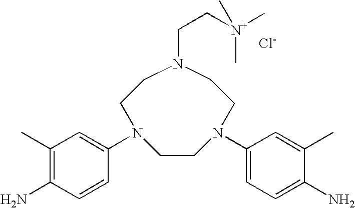 | [2-[4,7-bis(4-amino- 3-methylphenyl)- [1,4,7]triazonan-1- yl]ethyl]trimethyl- ammonium chloride |
| 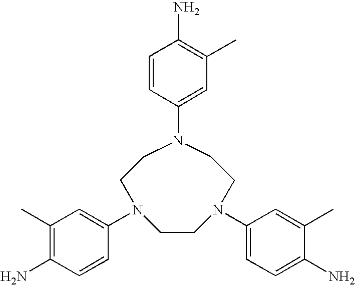 | 1,4,7-tris(4-amino-3- methylphenyl)[1,4,7]- triazonane |
| 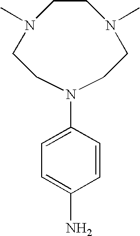 | 1,4-dimeethyl-7-(4- aminophenyl)[1,4,7]- triazonane |
| 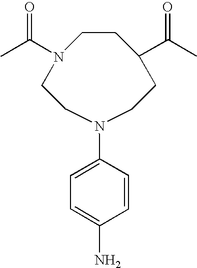 | 1,4-diacetyl-7-(4- aminophenyl)[1,4,7]- triazonane |
| 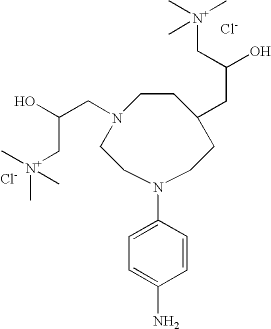 | 1-(4-aminophenyl)- 4,7-bis(γ-trimethyl- ammonium-β-hydroxy- propyl)[1,4,7]tri- azonane |
| 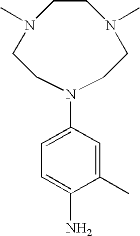 | 1,4-dimethyl-7-(4- amino-3-methylphen- yl)[1,4,7]triazonane |
| 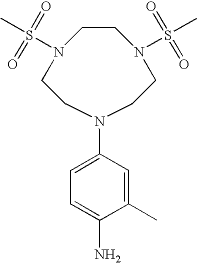 | 1,4-bis(methanesulf- onyl)-7-(4-amino-3- methylphenyl)[1,4,7]- triazonane |
| 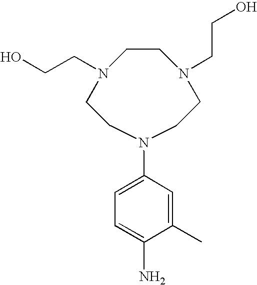 | 1,4-bis(β-hydroxy- ethyl)-7-(4-amino-3- methylphenyl)[1,4,7]- triazonane |
| 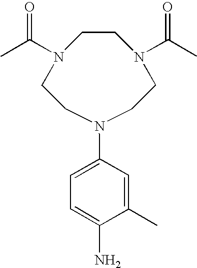 | 1,4-diacetyl-7-(4- amino-3-methylphen- yl)[1,4,7]triazonane |
| 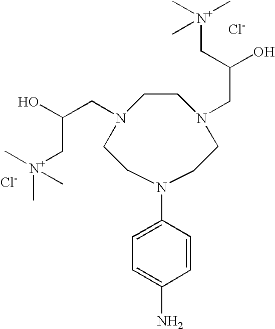 | 1,4-bis(γ-trimethyl- ammonium-β-hydroxy- propyl)-7-(4-amino-3- methylphenyl)[1,4,7]- triazonane chloride |

Among these compounds, the derivatives of formula (I) that are particularly preferred are 1-(4-aminophenyl)[1,4,7]triazonane; 1,4-bis(4-aminophenyl)-[1,4,7]triazonane; 1,4,7-tris(4-aminophenyl)[1,4,7]-triazonane; 2-[4,7-bis(4-aminophenyl)[1,4,7]triazonan-1-yl]ethanol and 3-{3-[4,7-bis(4-aminophenyl)[1,4,7]-triazonan-1-yl]propyl}-1-methyl-3H-imidazol-1-ium chloride.

The carbon substituted with R₄ may be of (R) and/or (S) configuration.

A subject of the present invention is also the use as an oxidation base for the oxidation dyeing of keratin fibres, and in particular of human keratin fibres such as the hair, of a triazacyclononane derivative as defined above.

A subject of the present invention is also a composition for dyeing keratin fibres, and in particular human keratin fibres such as the hair, comprising, in a medium that is suitable for dyeing, at least one triazacyclononane derivative as defined above as oxidation base.

The triazacyclononane derivatives in accordance with the invention are each generally present in an amount of between 0.001% and 10% by weight approximately relative to the total weight of the dye composition, and preferably between 0.005% and 6%.

The composition of the present invention may also comprise one or more additional oxidation bases conventionally used in oxidation dyeing, other than those described above. By way of example, these additional oxidation bases are chosen from para-phenylenediamines other than the triazacyclononane derivatives according to the invention: bis(phenyl)alkylenediamines, para-aminophenols, bis-para-aminophenols, ortho-aminophenols, ortho-phenylenediamines and heterocyclic bases, and the addition salts thereof.

Among the para-phenylenediamines that may be mentioned, for example, are para-phenylenediamine; para-tolylenediamine; 2-chloro-para-phenylenediamine; 2,3-dimethyl-para-phenylenediamine; 2,6-dimethyl-para-phenylenediamine; 2,6-diethyl-para-phenylenediamine; 2,5-dimethyl-para-phenylenediamine; N,N-dimethyl-para-phenylenediamine; N,N-diethyl-para-phenylenediamine; N,N-dipropyl-para-phenylenediamine; 4-amino-N,N-diethyl-3-methylaniline; N,N-bis(β-hydroxyethyl)-para-phenylenediamine; 4-N,N-bis(β-hydroxyethyl)amino-2-methylaniline; 4-N,N-bis(β-hydroxyethyl)amino-2-chloroaniline; 2-β-hydroxyethyl-para-phenylenediamine; 2-fluoro-para-phenylenediamine; 2-isopropyl-para-phenylenediamine; N-(β-hydroxypropyl)-para-phenylenediamine; 2-hydroxymethyl-para-phenylenediamine; N,N-dimethyl-3-methyl-para-phenylenediamine; N-ethyl-N-(β-hydroxyethyl)-para-phenylenediamine; N-(β,γ-dihydroxypropyl)-para-phenylenediamine; N-(4′-aminophenyl)-para-phenylenediamine; N-phenyl-para-phenylenediamine; 2-(β-hydroxyethyloxy)-para-phenylenediamine; 2-(β-acetylaminoethyloxy)-para-phenylenediamine; N-(β-methoxyethyl)-para-phenylenediamine; 4-aminophenylpyrrolidine; 2-thienyl-para-phenylenediamine; 2-(β-hydroxyethylamino)-5-aminotoluene and 3-hydroxy-1-(4′-aminophenyl)-pyrrolidine, and the addition salts thereof with an acid.

Among the para-phenylenediamines mentioned above, para-phenylenediamine; para-tolylenediamine; 2-isopropyl-para-phenylenediamine; 2-(β-hydroxyethyl)-para-phenylenediamine; 2-(β-hydroxyethyloxy)-para-phenylenediamine; 2,6-dimethyl-para-phenylenediamine; 2,6-diethyl-para-phenylenediamine; 2,3-dimethyl-para-phenylenediamine; N,N-bis(β-hydroxyethyl)-para-phenylenediamine; 2-chloro-para-phenylenediamine and 2-(β-acetylaminoethyloxy)-para-phenylenediamine, and the addition salts thereof with an acid are particularly preferred.

Among the bis(phenyl)alkylenediamines that may be mentioned, for example, are N,N′-bis(β-hydroxyethyl)-N,N′-bis(4′-aminophenyl)-1,3-diaminopropanol; N,N′-bis(β-hydroxyethyl)-N,N′-bis(4′-aminophenyl)ethylenediamine; N,N′-bis(4-aminophenyl)tetramethylenediamine; N,N′-bis(β-hydroxyethyl)-N,N′-bis(4-aminophenyl)tetramethylenediamine; N,N′-bis(4-methylaminophenyl)tetramethylenediamine; N,N′-bis(ethyl)-N,N′-(4′-amino-3′-methylphenyl)ethylenediamine and 1,8-bis(2,5-diaminophenoxy)-3,6-dioxaoctane, and the addition salts thereof with an acid.

Among the para-aminophenols that may be mentioned, for example, are para-aminophenyl; 4-amino-3-methylphenol; 4-amino-3-fluorophenol; 4-amino-3-hydroxymethylphenol; 4-amino-2-methylphenol; 4-amino-2-hydroxymethylphenol; 4-amino-2-methoxymethylphenol; 4-amino-2-aminomethylphenol; 4-amino-2-(β-hydroxyethylaminomethyl)phenol and 4-amino-2-fluorophenol, and the addition salts thereof with an acid.

Among the ortho-aminophenols that may be mentioned, for example, are 2-aminophenol; 2-amino-5-methylphenol; 2-amino-6-methylphenol and 5-acetamido-2-aminophenol, and the addition salts thereof with an acid.

Among the heterocyclic bases, mention may be made, for example, of pyridine derivatives, pyrimidine derivatives and pyrazole derivatives.

Among the pyridine derivatives that may be mentioned are the compounds described, for example, in patents GB 1 026 978 and GB 1 153 196, such as 2,5-diaminopyridine; 2-(4-methoxyphenyl)amino-3-aminopyridine; 2,3-diamino-6-methoxypyridine; 2-(β-methoxyethylamino)-3-amino-6-methoxypyridine and 3,4-diaminopyridine, and the addition salts thereof with an acid.

Other pyridine oxidation bases that are useful in the present invention are the 3-aminopyrazolo[1,5-a]pyridine oxidation bases or the addition salts thereof described, for example, in patent application FR 2 801 308. By way of example, mention may be made of pyrazolo[1,5-a]pyrid-3-ylamine; 2-acetylaminopyrazolo[1,5-a]pyrid-3-ylamine; 2-morpholin-4-ylpyrazolo[1,5-a]pyrid-3-ylamine; 3-aminopyrazolo[1,5-a]pyridine-2-carboxylic acid; 2-methoxypyrazolo[1,5-a]pyrid-3-ylamine; (3-aminopyrazolo-[1,5-a]pyrid-7-yl)methanol; 2-(3-aminopyrazolo[1,5-a]-pyrid-5-yl)ethanol; 2-(3-aminopyrazolo[1,5-a]pyrid-7-yl)ethanol; (3-aminopyrazolo[1,5-a]pyrid-2-yl)methanol; 3,6-diaminopyrazolo[1,5-a]pyridine; 3,4-diaminopyrazolo[1,5-a]pyridine; pyrazolo[1,5-a]pyridine-3,7-diamine; 7-morpholin-4-ylpyrazolo[1,5-a]pyrid-3-ylamine; pyrazolo[1,5-a]pyridine-3,5-diamine; 5-morpholin-4-ylpyrazolo[1,5-a]pyrid-3-ylamine; 2-[(3-aminopyrazolo[1,5-a]pyrid-5-yl)(2-hydroxyethyl)amino]ethanol; 2-[(3-aminopyrazolo[1,5-a]pyrid-7-yl)-(2-hydroxyethyl)amino]ethanol; 3-aminopyrazolo-[1,5-a]pyrid-5-ol; 3-aminopyrazolo[1,5-a]pyrid-4-ol; 3-aminopyrazolo[1,5-a]pyrid-6-ol; 3-aminopyrazolo-[1,5-a]pyrid-7-ol and also the addition salts thereof with an acid or with a base.

Among the pyrimidine derivatives that may be mentioned are the compounds described, for example, in patent DE 2 359 399 or patents JP 88-169 571; JP 05 63 124; EP 0 770 375 or patent application WO 96/15765, such as 2,4,5,6-tetraminopyrimidine; 4-hydroxy-2,5,6-triaminopyrimidine; 2-hydroxy-4,5,6-triaminopyrimidine; 2,4-dihydroxy-5,6-diaminopyrimidine and 2,5,6-triaminopyrimidine, and pyrazolopyrimidine derivatives such as those mentioned in patent application FR-A-2 750 048 and among which mention may be made of pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol; 2-(7-aminopyrazolo[1,5-a]pyrimidin-3-ylamino)ethanol; 2-[(3-aminopyrazolo[1,5-a]pyrimidin-7-yl)(2-hydroxyethyl)amino]ethanol; 2-[(7-aminopyrazolo-[1,5-a]pyrimidin-3-yl)(2-hydroxyethyl)amino]ethanol; 5,6-dimethylpyrazolo[1,5-a]pyrimidine-3,7-diamine; 2,6-dimethylpyrazolo[1,5-a]pyrimidine-3,7-diamine; 2,5,N7,N7-tetramethylpyrazolo[1,5-a]pyrimidine-3,7-diamine and 3-amino-5-methyl-7-imidazolylpropylaminopyrazolo[1,5-a]pyrimidine, and the addition salts thereof with an acid and the tautomeric forms thereof, when a tautomeric equilibrium exists.

Among the pyrazole derivatives that may be mentioned are the compounds described in patents DE 3 843 892 and DE 4 133 957 and patent applications WO 94/08969, WO 94/08970, FR-A-2 733 749 and DE 195 43 988, such as 4,5-diamino-1-methylpyrazole; 4,5-diamino-1-(β-hydroxyethyl)pyrazole; 3,4-diaminopyrazole; 4,5-diamino-1-(4′-chlorobenzyl)pyrazole; 4,5-diamino-1,3-dimethylpyrazole; 4,5-diamino-3-methyl-1-phenylpyrazole; 4,5-diamino-1-methyl-3-phenylpyrazole; 4-amino-1,3-dimethyl-5-hydrazinopyrazole; 1-benzyl-4,5-diamino-3-methylpyrazole; 4,5-diamino-3-tert-butyl-1-methylpyrazole; 4,5-diamino-1-tert-butyl-3-methylpyrazole; 4,5-diamino-1-(β-hydroxyethyl)-3-methylpyrazole; 4,5-diamino-1-ethyl-3-methylpyrazole; 4,5-diamino-1-ethyl-3-(4′-methoxyphenyl)pyrazole; 4,5-diamino-1-ethyl-3-hydroxymethylpyrazole; 4,5-diamino-3-hydroxymethyl-1-methylpyrazole; 4,5-diamino-3-hydroxymethyl-1-isopropylpyrazole; 4,5-diamino-3-methyl-1-isopropylpyrazole; 4-amino-5-(2′-aminoethyl)amino-1,3-dimethylpyrazole; 3,4,5-triaminopyrazole; 1-methyl-3,4,5-triaminopyrazole; 3,5-diamino-1-methyl-4-methylaminopyrazole and 3,5-diamino-4-(β-hydroxyethyl)amino-1-methylpyrazole, and the addition salts thereof with an acid.

When additional oxidation bases are present in the composition of the invention, the amount of each of them is generally between 0.001% and 10% by weight approximately relative to the total weight of the dye composition, and preferably between 0.005% and 6%.

In general, the addition salts of the oxidation bases and of the couplers that may be used in the context of the invention are chosen especially from the addition salts with an acid, such as the hydrochlorides, hydrobromides, sulfates, citrates, succinates, tartrates, lactates, tosylates, benzenesulfonates, phosphates and acetates, and the addition salts with a base, such as sodium hydroxide, potassium hydroxide, ammonia, amines or alkanolamines.

The dye composition in accordance with the invention may also contain one or more direct dyes that may be chosen especially from nitrobenzene dyes, azo direct dyes and methine direct dyes. These direct dyes may be of nonionic, anionic or cationic nature.

The medium that is suitable for dyeing, also known as the dye support, is a cosmetic medium that generally consists of water or a mixture of water and at least one organic solvent to dissolve the compounds that would not be sufficiently soluble in water. As organic solvent, mention may be made, for example, of C₁-C₄ lower alkanols, such as ethanol and isopropanol; polyols and polyol ethers such as 2-butoxyethanol, propylene glycol, propylene glycol monomethyl ether, diethylene glycol monoethyl ether and monomethyl ether, as well as aromatic alcohols such as benzyl alcohol or phenoxyethanol, and mixtures thereof.

The solvents are present in proportions preferably of between 1% and 40% by weight approximately relative to the total weight of the dye composition, and even more preferably between 5% and 30% by weight approximately.

The dye composition in accordance with the invention can also contain various adjuvants conventionally used in compositions for dyeing the hair, such as anionic, cationic, nonionic, amphoteric or zwitterionic surfactants or mixtures thereof, anionic, cationic, nonionic, amphoteric or zwitterionic polymers or mixtures thereof, inorganic or organic thickeners, and in particular anionic, cationic, nonionic or amphoteric associative polymeric thickeners, antioxidants, penetration agents, sequestering agents, fragrances, buffers, dispersing agents, packaging agents such as, for example, silicones, which may or may not be volatile or modified, film-forming agents, ceramides, preserving agents and opacifiers.

The above adjuvants are generally present in an amount for each of them of between 0.01% and 20% by weight relative to the weight of the composition.

Needless to say, a person skilled in the art will take care to select this or these optional additional compounds such that the advantageous properties intrinsically associated with the oxidation dye composition in accordance with the invention are not, or are not substantially, adversely affected by the addition(s) envisaged.

The pH of the dye composition in accordance with the invention is generally between about 3 and 12 and preferably between about 5 and 11. It may be adjusted to the desired value using acidifying or basifying agents usually used in the dyeing of keratin fibres, or alternatively using standard buffer systems.

Among the acidifying agents that may be mentioned, for example, are inorganic or organic acids such as hydrochloric acid, orthophosphoric acid, sulfuric acid, carboxylic acids such as acetic acid, tartaric acid, citric acid and lactic acid, and sulfonic acids.

Among the basifying agents that may be mentioned, for example, are aqueous ammonia, alkaline carbonates, alkanolamines such as mono-, di- and triethanolamine and derivatives thereof, sodium hydroxide, potassium hydroxide and the compounds of formula (III) below: 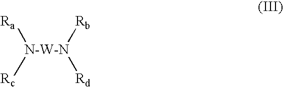
in which W is a propylene residue that is unsubstituted or substituted with a hydroxyl group or a C₁-C₄ alkyl radical; Rₐ, R_{b}, R_{c} and R_{d}, which may be identical or different, represent a hydrogen atom, a C₁-C₄ alkyl radical or a C₁-C₄ hydroxyalkyl radical.

The dye composition according to the invention may be in various forms, such as in the form of liquids, creams or gels, or in any other form that is suitable for dyeing keratin fibres, and especially human hair.

The process of the present invention is a process in which the composition according to the present invention as described above is applied to the fibres and the colour revealed using an oxidizing agent. The colour may be developed at acidic, neutral or alkaline pH and the oxidizing agent may be added to the composition of the invention just at the time of use, or it may be used starting with an oxidizing composition containing it, which is applied simultaneously or sequentially to the composition of the invention.

According to one particular embodiment, the composition according to the present invention is mixed, preferably at the time of use, with a composition containing, in a medium that is suitable for dyeing, at least one oxidizing agent, this oxidizing agent being present in an amount that is sufficient to develop a coloration. The mixture obtained is then applied to the keratin fibres. After an action time of 3 to 50 minutes approximately, preferably 5 to 30 minutes approximately, the keratin fibres are rinsed, washed with shampoo, rinsed again and then dried.

The oxidizing agents conventionally used for the oxidation dyeing of keratin fibres are, for example, hydrogen peroxide, urea peroxide, alkali metal bromates, persalts such as perborates and persulfates, peracids and oxidase enzymes, among which mention may be made of peroxidases, 2-electron oxidoreductases such as uricases, and 4-electron oxygenases, for instance laccases. Hydrogen peroxide is particularly preferred.

The oxidizing composition may also contain various adjuvants conventionally used in compositions for dyeing the hair and as defined above.

The pH of the oxidizing composition containing the oxidizing agent is such that, after mixing with the dye composition, the pH of the resulting composition applied to the keratin fibres ranges preferably between 3 and 12 approximately and even more preferably between 5 and 11. It may be adjusted to the desired value by means of acidifying or basifying agents usually used in the dyeing of keratin fibres and as defined above.

The ready-to-use composition that is finally applied to the keratin fibres may be in various forms, such as in the form of liquids, creams or gels or any other form that is suitable for dyeing keratin fibres, and especially human hair.

Another subject of the invention is a multi-compartment dyeing device or “kit”, in which a first compartment contains the above-defined dye composition of the present invention and a second compartment contains an oxidizing agent. This device may be equipped with a means for applying the desired mixture to the hair, such as the devices described in patent FR-2 586 913 in the name of the Applicant.

Using this device, it is possible to dye keratin fibres using a process that includes mixing a dye composition comprising at least one triazacyclononane derivative according to the invention with an oxidizing agent, and applying the mixture obtained to the keratin fibres for a time that is sufficient to develop the desired coloration.

The triazacyclononane derivatives in accordance with the invention may be obtained by nucleophilic reaction of the triazacyclononane ring with one, two or three para-fluoronitrobenzene molecules, optionally followed by functionalization of the remaining nitrogen atoms on the triazacyclononane ring, then followed by reduction of the nitro groups to amine. In particular, they may be obtained by analogy with preparation processes described in the literature: see especially patent application DE 42 41 532. The reduction of the nitro groups to amine may be performed in the presence of zinc: see especially the reference J. Heterocycl. Chem. 1992, 29 (6), 1513-1517.

Finally, a subject of the invention is intermediate triazacyclononane derivatives substituted on at least one of the nitrogen atoms with a substituted or unsubstituted 4′-nitrophenyl group.

According to one particular embodiment of the invention, the intermediate triazacyclononane derivatives substituted on at least one of the nitrogen atoms with a substituted or unsubstituted 4′-nitrophenyl group are chosen from the compounds of formula (IV) below, and the addition salts thereof: 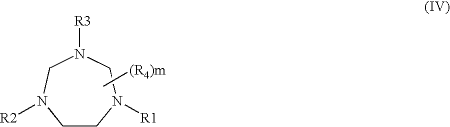
in which:
R₁, R₂ and R₃ represent:
   a hydrogen atom;
   a saturated or unsaturated alkyl radical, which is unsubstituted or substituted with one or more carboxyl, alkylcarbonyl, alkoxycarbonyl, carbamoyl, mono- or dialkylcarbamoyl, alkylsulfonyl, trialkylammonium or N-alkylimidazolium radicals, or saturated or unsaturated heterocyclic radicals containing 4, 5, 6 or 7 atoms, the hetero atom(s) of which is (are) chosen from nitrogen, oxygen and sulfur;
   a radical —CH₂—R′ in which R′ is a saturated or unsaturated alkyl radical substituted with one or more hydroxyl, alkoxy, amino, mono- or dialkylamino, trialkylammonium, alkylcarbonylamino or alkylcarbonyloxy radicals;
   a saturated or unsaturated alkylcarbonyl radical, which is unsubstituted or substituted with one or more hydroxyl, alkoxy, amino, mono- or dialkylamino, alkylsulfonyl, carboxyl, alkylcarbonyl, alkoxycarbonyl, carbamoyl, mono- or dialkylcarbamoyl, trialkylammonium or N-alkylimidazolium radicals, or saturated or unsaturated heterocyclic radicals containing 4, 5, 6 or 7 atoms, the hetero atom(s) of which is (are) chosen from nitrogen, oxygen and sulfur;
   an alkoxycarbonyl radical;
   a mono- or dialkylcarbamoyl radical;
   a carbamoyl radical;
   a carboxyl radical;
   an alkylsulfonyl radical;
   an arylsulfonyl radical;
   a radical corresponding to formula (V): 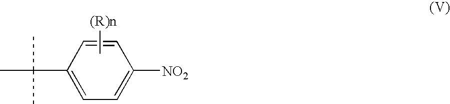
      in which:
R represents:
   a saturated or unsaturated alkyl radical, which is unsubstituted or substituted with one or more hydroxyl, amino, guanidinyl, trialkylammonium, N-alkylimidazolium, carboxyl or carbamoyl radicals;
   a saturated or unsaturated alkoxy radical, which is unsubstituted or substituted with one or more hydroxyl, amino, trialkylammonium or N-alkylimidazolium radicals;
   an alkoxyalkyl radical;
   a hydroxyalkoxyalkyl radical;
   an aminoalkyl radical, the amine possibly being unsubstituted or mono- or disubstituted with an alkyl, acetyl or hydroxyalkyl radical;
n is between 0 and 4, it being understood that when n is greater than 1, then the radicals R may be identical or different;
   it being understood that at least one of the radicals R₁, R₂ and R₃ is chosen from the radicals of formula (II);
R₄ represents:
   a saturated or unsaturated alkyl radical;
   a hydroxyalkyl radical;
   an alkoxyalkyl radical;
   an alkylcarbonyl radical;
   a hydroxyalkoxyalkyl radical;
   an aminoalkyl radical, the amine possibly being unsubstituted or mono- or disubstituted with an alkyl, acetyl or hydroxyalkyl radical;
   a hydroxyl or aminoalkyl radical;
   a carboxyl radical;
   a carboxyalkyl radical;
   a carbamoyl radical;
   a carbamoylalkyl radical;
   an alkoxycarbonyl radical;
   a mono- or dialkylaminocarbonyl radical;
m is between 0 and 6, it being understood that when m is greater than 1, then the radicals R₄ may be identical or different.

The examples that follow serve to illustrate the invention without, however, being limiting in nature.

### EXAMPLES

### Example 1

### Synthesis of 1-(4-aminophenyl)[1,4,7]triazonane (2)

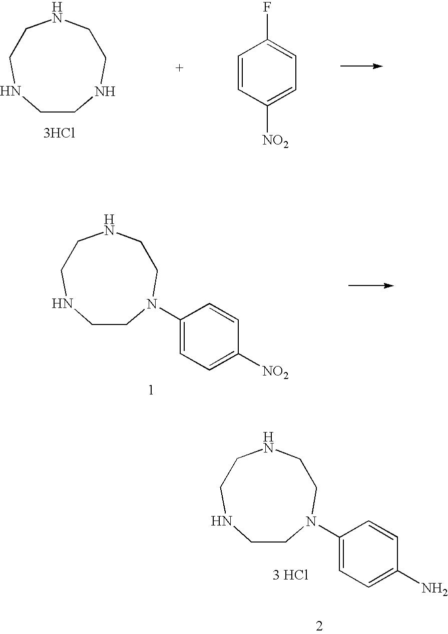

### Preparation of 1-(4-nitrophenyl)[1,4,7]triazonane (1)

1 g of triazacyclononane trihydrochloride (4.2 mmol) and 0.4 g of sodium hydride (0.01 mmol) are placed in a round-bottomed flask containing 15 ml of anhydrous tetrahydrofuran. 0.296 g of 1-fluoro-4-nitrobenzene (2.1 mmol) is added to this mixture at room temperature. After reaction for 6 hours, the excess sodium hydride is hydrolysed with water and the THF is evaporated off. The aqueous phase is then extracted with dichloromethane. The organic phases are combined, dried with magnesium sulfate and then evaporated. The crude product obtained is recrystallized from dichloromethane to give 0.03 g of a yellow solid, i.e. a yield of 3%. The results of the analyses obtained by ¹H NMR and by mass spectrometry are as follows:

¹H NMR (400 MHz, DMSO): 2.72 (s, 4H); 3.1 (m, 4H); 3.76 (m, 4H); 6.5 (s, 2H); 6.85 (d, 2H); 8.09 (d, 2H).

Mass (ESI+): 251 (MH+).

### Preparation of 1-(4-aminophenyl)[1,4,7]triazonane (2)

The 1-(4-nitrophenyl)[1,4,7]triazonane (1) obtained above is added to 10 ml of ethanol and reduced in the presence of zinc and ammonium chloride. The 1-(4-aminophenyl)[1,4,7]triazonane (2) obtained is isolated in the form of the hydrochloride.

### Example 2

### Synthesis of 1,4-bis(4-aminophenyl)[1,4,7-triazonane (4)

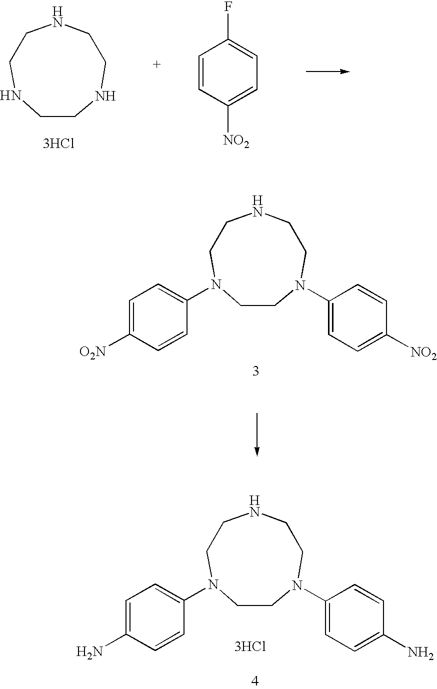

### Preparation of 1,4-bis(4-nitrophenyl)[1,4,7]triazonane (3)

20 g of triazacyclononane trihydrochloride (83.8 mmol), 35.4 g of 1-fluoro-4-nitrobenzene (251.4 mmol) and 40 g of potassium carbonate (290 mmol) are placed in 150 ml of water and the mixture is refluxed with stirring for 24 hours. After cooling to room temperature, the orange-yellow solid obtained is filtered off and rinsed with water and then with petroleum ether. After drying, 31 g of product are obtained, i.e. a yield of 99%.

The results of the analyses obtained by ¹H NMR and by mass spectrometry are as follows:

¹H NMR (400 MHz, DMSO): 2.2 (m, 1H); 2.8 (m, 4H); 3.4 (m, 4H); 3.9 (m, 4H); 6.8 (m, 4H); 8.1 (m, 4H).

Mass (ESI+): m/z=372 (MH+).

### Preparation of 1,4-bis(4-aminophenyl)[1,4,7]triazonane Hydrochloride (4)

2 g of 1,4-bis(4-nitrophenyl)[1,4,7]triazonane (3) obtained above (5.3 mmol) and 0.5 g of palladium on charcoal suspended in 450 ml of ethanol are placed in a hydrogenator. The hydrogenation is performed at 80° C. under a hydrogen pressure of 10 bar. After cooling to room temperature and filtering off the catalyst, 2 g of product are isolated in the form of the hydrochloride, i.e. a yield of 80%.

The results of the analyses obtained by ¹H NMR and by mass spectrometry are as follows:

¹H NMR (400 MHz, D₂O): 3.3 (m, 4H); 3.7 (m, 8H); 6.9 (m, 4H); 7.3 (m, 4H).

Mass (ESI+): m/z=312 (MH+).

### Example 3

### Synthesis of 1,4,7-tris(4-aminophenyl)-[1,4,7]triazacyclononane (6)

(6)

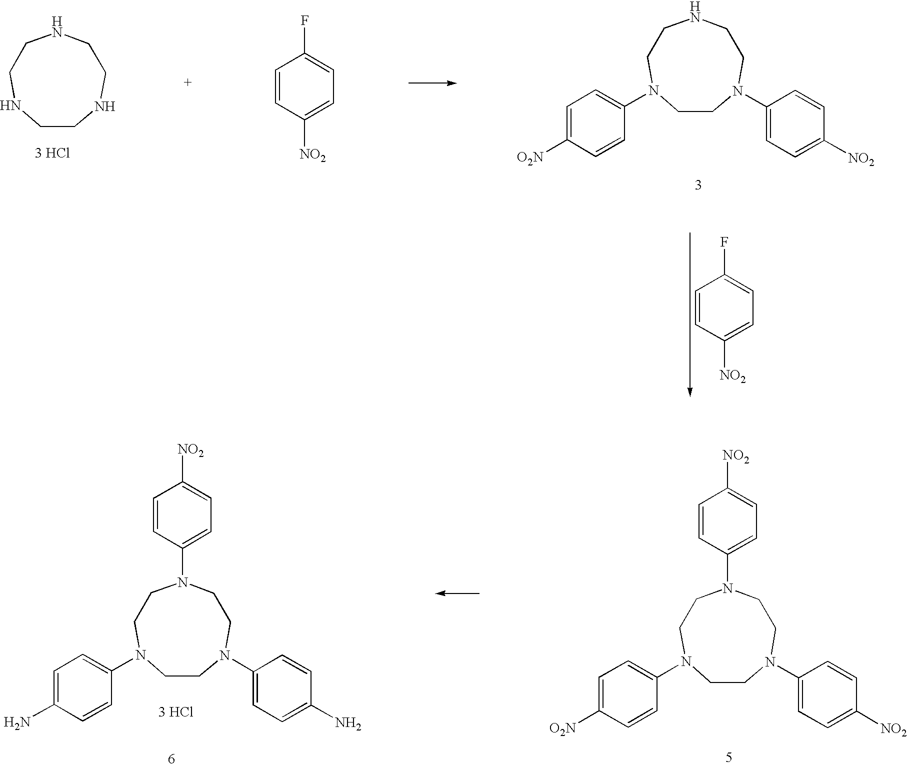

### Preparation of 1,4,7-tris(4-nitrophenyl)[1,4,7]triazacyclononane (5)

5 g of 1,4-bis(4-nitrophenyl)[1,4,7]triazonane (3) obtained above (0.013 mol), 2.28 g of para-fluoronitrobenzene (0.016 mol) and 2.24 g of potassium carbonate (0.016 mol) are placed in 25 ml of N-methylpyrrolidinone and the mixture is maintained at 120° C. for 13 hours. The reaction medium is poured into saturated aqueous sodium chloride solution to give a yellow precipitate. 0.3 g of product is obtained by purification by chromatography, i.e. a yield of 5%.

The results of the analyses obtained by ¹H NMR and by mass spectrometry are as follows:

¹H NMR (400 MHz, DMSO): 3.75 (s, 12H); 6.76 (m, 6H); 8 (m, 6H).

Mass (chemical ionization NH₃): 493 (MH⁺) and 510 (MNH₄⁺).

### Preparation of 1,4,7-tris(4-aminophenyl)[1,4,7]triazonane (6)

The 1,4,7-tris(4-nitrophenyl)[1,4,7]triazacyclononane (5) obtained above is added to 300 ml of ethanol and reduced in the presence of zinc and ammonium chloride. The 1,4,7-tris(4-aminophenyl)-[1,4,7]triazonane (6) obtained is isolated in the form of the hydrochloride.

### Example 4

### Synthesis of 2-[4,7-bis(4-aminophenyl)-[1,4,7]triazonan-1-yl]ethanol (8)

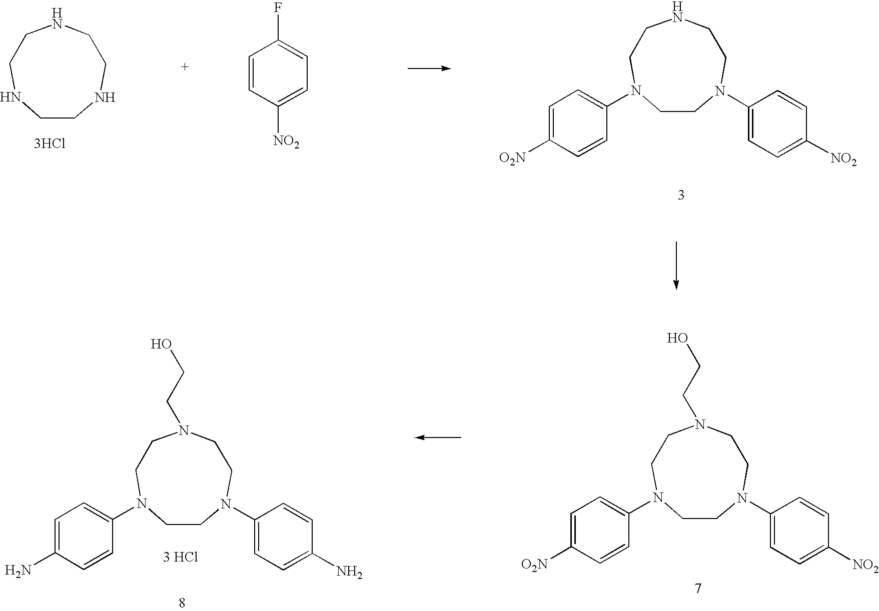

### Preparation of 2-[4,7-bis(4-nitrophenyl)[1,4,7]triazonan-1-yl]ethanol (7)

5 g of 1,4-bis(4-nitrophenyl)[1,4,7]triazonane (3) obtained above (13.46 mmol) and 2.8 ml of triethylamine (20.2 mmol) are placed in 13 ml of NMP. 1.43 ml of bromoethanol (20.2 mmol) are added to this mixture. The reaction medium thus obtained is heated at 110° C. for 7 hours. Water is added with stirring and the precipitate formed is filtered off and then rinsed thoroughly with water, and then with diisopropyl ether. After drying, 5.6 g of product are obtained, i.e. a yield of about 100%.

The results of the analyses obtained by ¹H NMR and by mass spectrometry are as follows:

¹H NMR (DMSO, 400 MHz): 2.59 (t, 2H); 2.7 (m, 4H); 3.29 (q+s, 2H+H₂O); 3.46 (m, 4H); 3.85 (s, 4H); 4.33 (t, 1H); 6.8 (m, 4H); 8.04 (m, 4H).

Mass (ESI+/− in MeOH/H₂O): m/Z=416 (M+H)⁺; 414 (M−H)⁻.

### Preparation of 2-[4,7-bis(4-aminophenyl)[1,4,7]triazonan-1-yl]ethanol (8)

5 g of 2-[4,7-bis(4-nitrophenyl)[1,4,7]-triazonan-1-yl]ethanol (7) obtained above (5.3 mmol) are placed in a hydrogenator in 50 ml of acetic acid and 100 ml of ethanol in the presence of 1 g of 5% palladium on charcoal. The hydrogenation is performed at 80° C. under a hydrogen pressure of 10 bar for four hours. After cooling to room temperature and filtering off the catalyst, 2.4 g of product are isolated in the form of the hydrochloride, i.e. a yield of 43%.

The results of the analyses obtained by ¹H NMR and by mass spectrometry are as follows:

¹H NMR (400 MHz, D₂O): 3.49 (t, 2H); 3.62-3.94 (t+m, 14H); 7.05-7.35 (2d, 8H).

Mass (ESI+): m/z=356 (MH+).

### Example 5

### Synthesis of 3-{3-[4,7-bis(4-aminophenyl)-[1,4,7]triazonan-1-yl]propyl}-1-methyl-3H-imidazol-1-ium Chloride (10)

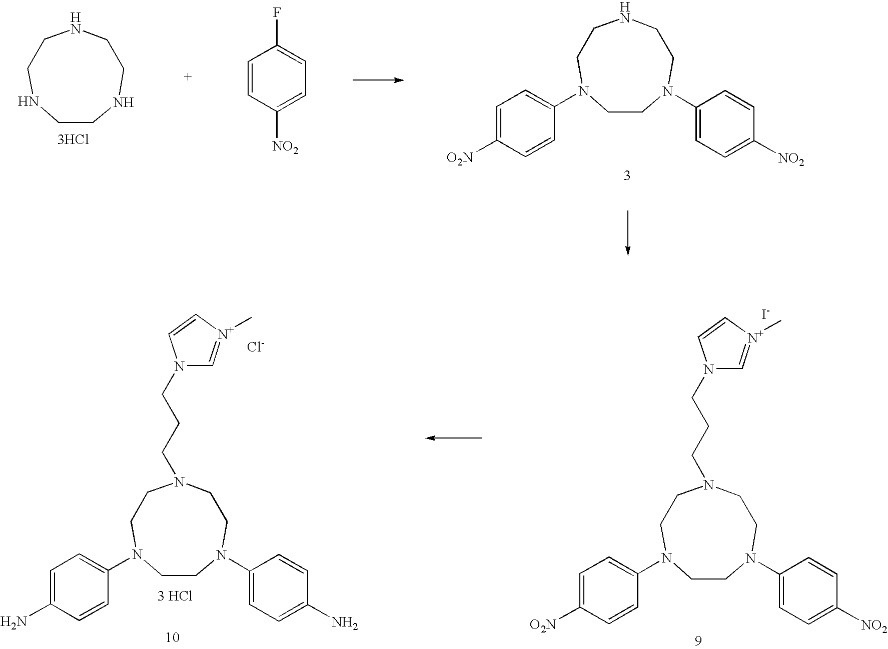

### Preparation of 3-{3-[4,7-bis(4-nitrophenyl)[1,4,7]-triazonan-1-yl]propyl}-1-methyl-3H-imidazol-1-ium (9)

0.5 g of 1,4-bis(4-nitrophenyl)[1,4,7]triazonane (3) obtained above (1.35 mmol) are placed in a 25 ml three-necked flask, followed by 5 ml of NMP, 0.19 ml of triethylamine (1.35 mmol) and finally the iodo derivative 3-(3-iodopropyl)-1-methyl-3H-imidazol-1-ium. The reaction medium is maintained at 120° C. for 17 hours. The cooled reaction medium is poured at room temperature into a solution of brine. A yellow gummy solid is isolated, washed with ethyl acetate and then with water to give a yellow powder.

The results of the analyses obtained by ¹H NMR and by mass spectrometry are as follows:

¹H NMR (DMSO, 400 MHz): 1.75 (m, 2H); 2.5 (t, 2H+DMSO); 2.68 (m, 4H); 3.48 (m, 4H); 3.83 (s, 3H); 3.84 (s, 4H); 4.01 (m, 2H); 6.81 (d, 4H); 7.63 (dd, 1H); 7.67 (dd, 1H); 8.05 (d, 4H); 9.02 (s, 1H).

Mass (ESI+/− in MeOH/H₂O): m/z=494 (M)+; 127 (I−).

### Preparation of 3-{3-[4,7-bis(4-aminophenyl)[1,4,7]-triazonan-1-yl]propyl}-1-methyl-3H-imidazol-1-ium (10)

3-{3-[4,7-Bis(4-nitrophenyl)[1,4,7]triazonan-1-yl]propyl}-1-methyl-3H-imidazol-1-ium (9) obtained above is added to 50 ml of ethanol and reduced in the presence of zinc and ammonium chloride. The 3-{3-[4,7-bis(4-aminophenyl)[1,4,7]triazonan-1-yl]propyl}-1-methyl-3H-imidazol-1-ium (10) obtained is isolated in the form of the hydrochloride.

### EXAMPLES OF DYEING

### Examples 1-14

### Dye Compositions Using 1-(4-aminophenyl)[1,4,7]triazonane (2)

### Examples 1 to 7

### Dyeing in Acidic Medium

The following dye compositions are prepared:

| | Example | | | | | | |
|---|---|---|---|---|---|---|---|
| | 1 | 2 | 3 | 4 | 5 | 6 | 7 |
| 1-(4-Aminophenyl)-[1,4,7]triazonane | 4 × 10⁻⁴ | 4 × 10⁻⁴ | 4 × 10⁻⁴ | 4 × 10⁻⁴ | 4 × 10⁻⁴ | 4 × 10⁻⁴ | 4 × 10⁻⁴ |
| | mol | mol | mol | mol | mol | mol | mol |
| Benzene-1,3-diol | 4 × 10⁻⁴ | — | — | — | — | — | — |
| | mol | | | | | | |
| 5-Amino-2-methylphenol | — | 4 × 10⁻⁴ | — | — | — | — | — |
| | | mol | | | | | |
| 1H-Indol-6-ol | — | — | 4 × 10⁻⁴ | — | — | — | — |
| | | | mol | | | | |
| 2-Aminopyridin-3-ol | — | — | — | 4 × 10⁻⁴ | — | — | — |
| | | | | mol | | | |
| 3,6-Dimethyl-1H-pyrazolo[5,1- | — | — | — | — | 4 × 10⁻⁴ | — | — |
| c]-[1,2,4]triazole | | | | | mol | | |
| 2-(2,4-Diamino-phenoxy)ethanol | — | — | — | — | — | 4 × 10⁻⁴ | — |
| hydrochloride | | | | | | mol | |
| 3-Amino-2-chloro-6-methylphenol | — | — | — | — | — | — | 4 × 10⁻⁴ |
| hydrochloride | | | | | | | mol |
| Dye support | (1) | (1) | (1) | (1) | (1) | (1) | (1) |
| Demineralized water qs | 100 g | 100 g | 100 g | 100 g | 100 g | 100 g | 100 g |

(1): pH 7 Dye Support

| | | |
|---|---|---|
| 96° ethyl alcohol | 20.8 | g |
| Sodium metabisulfite as an aqueous | 0.23 | g A.M. |
| 35% solution | | |
| Pentasodium salt of | 0.48 | g A.M. |
| diethylenetriaminepentaacetic acid | | |
| as an aqueous 40% solution | | |
| C₈-C₁₀ alkyl polyglucoside as an | 3.6 | g A.M. |
| aqueous 60% solution | | |
| Benzyl alcohol | 2.0 | g |
| Polyethylene glycol containing | 3.0 | g |
| 8 units of ethylene oxide | | |
| Na₂HPO₄ | 0.28 | g |
| KH₂PO₄ | 0.46 | g |

At the time of use, each composition is mixed with an equal weight of 20-volumes aqueous hydrogen peroxide solution (6% by weight). A final pH of 7 is obtained.

Each mixture obtained is applied to locks of grey hair containing 90% white hairs. After leaving to act for 30 minutes, the locks are rinsed, washed with a standard shampoo, rinsed again and then dried.

| | Example | | | | | | |
|---|---|---|---|---|---|---|---|
| | 1 | 2 | 3 | 4 | 5 | 6 | 7 |
| Shade | Yellow | Brown | Brown | Brown | Yellow | Blue-green | Violet- |
| observed | brown | | | | brown | grey | grey |

### Examples 8 to 14

### Dyeing in Basic Medium

The following dye compositions are prepared:

| | Example | | | | | | |
|---|---|---|---|---|---|---|---|
| | 8 | 9 | 10 | 11 | 12 | 13 | 14 |
| 1-(4-Aminophen-yl)[1,4,7]triazonane | 4 × 10⁻⁴ | 4 × 10⁻⁴ | 4 × 10⁻⁴ | 4 × 10⁻⁴ | 4 × 10⁻⁴ | 4 × 10⁻⁴ | 4 × 10⁻⁴ |
| | mol | mol | mol | mol | mol | mol | mol |
| Benzene-1,3-diol | 4 × 10⁻⁴ | — | — | — | — | — | — |
| | mol | | | | | | |
| 5-Amino-2-methylphenol | — | 4 × 10⁻⁴ | — | — | — | — | — |
| | | mol | | | | | |
| 1H-Indol-6-ol | — | — | 4 × 10⁻⁴ | — | — | — | — |
| | | | mol | | | | |
| 2-Aminopyridin-3-ol | — | — | — | 4 × 10⁻⁴ | — | — | — |
| | | | | mol | | | |
| 3,6-Dimethyl-1H-pyrazolo- | — | — | — | — | 4 × 10⁻⁴ | — | — |
| [5,1-c][1,2,4]-triazole | | | | | mol | | |
| 2-(2,4-Diamino-phenoxy)ethanol | — | — | — | — | — | 4 × 10⁻⁴ | — |
| hydrochloride | | | | | | mol | |
| 3-Amino-2-chloro-6-methylphenol | — | — | — | — | — | — | 4 × 10⁻⁴ |
| hydrochloride | | | | | | | mol |
| Dye support | (2) | (2) | (2) | (2) | (2) | (2) | (2) |
| Demineralized water qs | 100 g | 100 g | 100 g | 100 g | 100 g | 100 g | 100 g |

(2): pH 9.5 Dye Support

| | | |
|---|---|---|
| 96° ethyl alcohol | 20.8 | g |
| Sodium metabisulfite as an aqueous | 0.23 | g A.M. |
| 35% solution | | |
| Pentasodium salt of | 0.48 | g A.M. |
| diethylenetriaminepentaacetic acid | | |
| as an aqueous 40% solution | | |
| C₈-C₁₀ alkyl polyglucoside as an | 3.6 | g A.M. |
| aqueous 60% solution | | |
| Benzyl alcohol | 2.0 | g |
| Polyethylene glycol containing | 3.0 | g |
| 8 units of ethylene oxide | | |
| NH₄Cl | 4.32 | g |
| Aqueous ammonia containing 20% NH₃ | 2.94 | g |

At the time of use, each composition is mixed with an equal weight of 20-volumes aqueous hydrogen peroxide solution (6% by weight). A final pH of 9.5 is obtained.

Each mixture obtained is applied to locks of grey hair containing 90% white hairs. After leaving to act for 30 minutes, the locks are rinsed, washed with a standard shampoo, rinsed again and then dried.

| | Example | | | | | | |
|---|---|---|---|---|---|---|---|
| | 8 | 9 | 10 | 11 | 12 | 13 | 14 |
| Shade | Orange- | Brown | Orange | Orange | Red | Blue- | Violet- |
| observed | yellow | | | | | grey | grey |

### Examples 15 to 28

### Dye Compositions Using 1,4-bis(4-aminophenyl)[1,4,7]triazonane (4)

### Examples 15 to 21

### Dyeing in Acidic Medium

The following dye compositions are prepared:

| | Example | | | | | | |
|---|---|---|---|---|---|---|---|
| | 15 | 16 | 17 | 18 | 19 | 20 | 21 |
| 1,4-Bis(4-amino-phenyl)[1,4,7]- | 4 × 10⁻⁴ | 4 × 10⁻⁴ | 4 × 10⁻⁴ | 4 × 10⁻⁴ | 4 × 10⁻⁴ | 4 × 10⁻⁴ | 4 × 10⁻⁴ |
| triazonane | mol | mol | mol | mol | mol | mol | mol |
| Benzene-1,3-diol | 4 × 10⁻⁴ | — | — | — | — | — | — |
| | mol | | | | | | |
| 5-Amino-2-methylphenol | — | 4 × 10⁻⁴ | — | — | — | — | — |
| | | mol | | | | | |
| 1H-Indol-6-ol | — | — | 4 × 10⁻⁴ | — | — | — | — |
| | | | mol | | | | |
| 2-Aminopyridin-3-ol | — | — | — | 4 × 10⁻⁴ | — | — | — |
| | | | | mol | | | |
| 3,6-Dimethyl-1H-pyrazolo[5,1-c]- | — | — | — | — | 4 × 10⁻⁴ | — | — |
| [1,2,4]triazole | | | | | mol | | |
| 2-(2,4-Diamino-phenoxy)ethanol | — | — | — | — | — | 4 × 10⁻⁴ | — |
| hydrochloride | | | | | | mol | |
| 3-Amino-2-chloro-6-methylphenol | — | — | — | — | — | — | 4 × 10⁻⁴ |
| hydrochloride | | | | | | | mol |
| Dye support | (1) | (1) | (1) | (1) | (1) | (1) | (1) |
| Demineralized water qs | 100 g | 100 g | 100 g | 100 g | 100 g | 100 g | 100 g |

(1): pH 7 Dye Support

| | | |
|---|---|---|
| 96° ethyl alcohol | 20.8 | g |
| Sodium metabisulfite as an aqueous | 0.23 | g A.M. |
| 35% solution | | |
| Pentasodium salt of | 0.48 | g A.M. |
| diethylenetriaminepentaacetic acid | | |
| as an aqueous 40% solution | | |
| C₈-C₁₀ alkyl polyglucoside as an | 3.6 | g A.M. |
| aqueous 60% solution | | |
| Benzyl alcohol | 2.0 | g |
| Polyethylene glycol containing | 3.0 | g |
| 8 units of ethylene oxide | | |
| Na₂HPO₄ | 0.28 | g |
| KH₂PO₄ | 0.46 | g |

At the time of use, each composition is mixed with an equal weight of 20-volumes aqueous hydrogen peroxide solution (6% by weight). A final pH of 7 is obtained.

Each mixture obtained is applied to locks of grey hair containing 90% white hairs. After leaving to act for 30 minutes, the locks are rinsed, washed with a standard shampoo, rinsed again and then dried.

| | Example | | | | | | |
|---|---|---|---|---|---|---|---|
| | 15 | 16 | 17 | 18 | 19 | 20 | 21 |
| Shade | Violet- | Violet- | Violet- | Violet- | Brown | Violet- | Violet- |
| observed | grey | grey | grey | grey | | grey | grey |

### Examples 22 to 28

### Dyeing in Basic Medium

The following dye compositions are prepared:

| | Example | | | | | | |
|---|---|---|---|---|---|---|---|
| | 22 | 23 | 24 | 25 | 26 | 27 | 28 |
| 1,4-Bis(4-amino-phenyl)[1,4,7]- | 4 × 10⁻⁴ | 4 × 10⁻⁴ | 4 × 10⁻⁴ | 4 × 10⁻⁴ | 4 × 10⁻⁴ | 4 × 10⁻⁴ | 4 × 10⁻⁴ |
| triazonane | mol | mol | mol | mol | mol | mol | mol |
| Benzene-1,3-diol | 4 × 10⁻⁴ | — | — | — | — | — | — |
| | mol | | | | | | |
| 5-Amino-2-methylphenol | — | 4 × 10⁻⁴ | — | — | — | — | — |
| | | mol | | | | | |
| 1H-Indol-6-ol | — | — | 4 × 10⁻⁴ | — | — | — | — |
| | | | mol | | | | |
| 2-Aminopyridin-3-ol | — | — | — | 4 × 10⁻⁴ | — | — | — |
| | | | | mol | | | |
| 3,6-Dimethyl-1H-pyrazolo[5,1-c]- | — | — | — | — | 4 × 10⁻⁴ | — | — |
| [1,2,4]triazole | | | | | mol | | |
| 2-(2,4-Diamino-phenoxy)ethanol | — | — | — | — | — | 4 × 10⁻⁴ | — |
| hydrochloride | | | | | | mol | |
| 3-Amino-2-chloro-6-methylphenol | — | — | — | — | — | — | 4 × 10⁻⁴ |
| hydrochloride | | | | | | | mol |
| Dye support | (2) | (2) | (2) | (2) | (2) | (2) | (2) |
| Demineralized water qs | 100 g | 100 g | 100 g | 100 g | 100 g | 100 g | 100 g |

(2): pH 9.5 Dye Support

| | | |
|---|---|---|
| 96° ethyl alcohol | 20.8 | g |
| Sodium metabisulfite as an aqueous | 0.23 | g A.M. |
| 35% solution | | |
| Pentasodium salt of | 0.48 | g A.M. |
| diethylenetriaminepentaacetic acid | | |
| as an aqueous 40% solution | | |
| C₈-C₁₀ alkyl polyglucoside as an | 3.6 | g A.M. |
| aqueous 60% solution | | |
| Benzyl alcohol | 2.0 | g |
| Polyethylene glycol containing | 3.0 | g |
| 8 units of ethylene oxide | | |
| NH₄Cl | 4.32 | g |
| Aqueous ammonia containing 20% NH₃ | 2.94 | g |

At the time of use, each composition is mixed with an equal weight of 20-volumes aqueous hydrogen peroxide solution (6% by weight). A final pH of 9.5 is obtained.

Each mixture obtained is applied to locks of grey hair containing 90% white hairs. After leaving to act for 30 minutes, the locks are rinsed, washed with a standard shampoo, rinsed again and then dried.

| | Example | | | | | | |
|---|---|---|---|---|---|---|---|
| | 22 | 23 | 24 | 25 | 26 | 27 | 28 |
| Shade observed | Brown | Violet | Red | Brown | Violet | Violet | Violet |

### Examples 29 to 42

### Dye Compositions Using 1,4,7-tris(4-aminophenyl)[1,4,7]triazonane (6)

### Examples 29 to 35

### Dyeing in Acidic Medium

The following dye compositions are prepared:

| | Example | | | | | | |
|---|---|---|---|---|---|---|---|
| | 29 | 30 | 31 | 32 | 33 | 34 | 35 |
| 1,4,7-Tris(4-aminophenyl)[1,4,7]- | 4 × 10⁻⁴ | 4 × 10⁻⁴ | 4 × 10⁻⁴ | 4 × 10⁻⁴ | 4 × 10⁻⁴ | 4 × 10⁻⁴ | 4 × 10⁻⁴ |
| triazonane | mol | mol | mol | mol | mol | mol | mol |
| Benzene-1,3-diol | 4 × 10⁻⁴ | — | — | — | — | — | — |
| | mol | | | | | | |
| 5-Amino-2-methylphenol | — | 4 × 10⁻⁴ | — | — | — | — | — |
| | | mol | | | | | |
| 1H-Indol-6-ol | — | — | 4 × 10⁻⁴ | — | — | — | — |
| | | | mol | | | | |
| 2-Aminopyridin-3-ol | — | — | — | 4 × 10⁻⁴ | — | — | — |
| | | | | mol | | | |
| 3,6-Dimethyl-1H-pyrazolo[5,1-c]- | — | — | — | — | 4 × 10⁻⁴ | — | — |
| [1,2,4]triazole | | | | | mol | | |
| 2-(2,4-Diamino-phenoxy)ethanol | — | — | — | — | — | 4 × 10⁻⁴ | — |
| hydrochloride | | | | | | mol | |
| 3-Amino-2-chloro-6-methylphenol | — | — | — | — | — | — | 4 × 10⁻⁴ |
| hydrochloride | | | | | | | mol |
| Dye support | (1) | (1) | (1) | (1) | (1) | (1) | (1) |
| Demineralized water qs | 100 g | 100 g | 100 g | 100 g | 100 g | 100 g | 100 g |

(1): pH 7 Dye Support

| | | |
|---|---|---|
| 96° ethyl alcohol | 20.8 | g |
| Sodium metabisulfite as an aqueous | 0.23 | g A.M. |
| 35% solution | | |
| Pentasodium salt of | 0.48 | g A.M. |
| diethylenetriaminepentaacetic acid | | |
| as an aqueous 40% solution | | |
| C₈-C₁₀ alkyl polyglucoside as an | 3.6 | g A.M. |
| aqueous 60% solution | | |
| Benzyl alcohol | 2.0 | g |
| Polyethylene glycol containing | 3.0 | g |
| 8 units of ethylene oxide | | |
| Na₂HPO₄ | 0.28 | g |
| KH₂PO₄ | 0.46 | g |

At the time of use, each composition is mixed with an equal weight of 20-volumes aqueous hydrogen peroxide solution (6% by weight). A final pH of 7 is obtained.

Each mixture obtained is applied to locks of grey hair containing 90% white hairs. After leaving to act for 30 minutes, the locks are rinsed, washed with a standard shampoo, rinsed again and then dried.

| | Example | | | | | | |
|---|---|---|---|---|---|---|---|
| | 29 | 30 | 31 | 32 | 33 | 34 | 35 |
| Shade | Yellow | Orange- | Yellow- | Yellow | Yellow | Blue-green | Yellow- |
| observed | | yellow | brown | | | grey | brown |

### Examples 36 to 42

### Dyeing in Basic Medium

The following dye compositions are prepared:

| | Example | | | | | | |
|---|---|---|---|---|---|---|---|
| | 36 | 37 | 38 | 39 | 40 | 41 | 42 |
| 1,4,7-Tris(4-aminophenyl)- | 4 × 10⁻⁴ | 4 × 10⁻⁴ | 4 × 10⁻⁴ | 4 × 10⁻⁴ | 4 × 10⁻⁴ | 4 × 10⁻⁴ | 4 × 10⁻⁴ |
| [1,4,7]triazonane | mol | mol | mol | mol | mol | mol | mol |
| Benzene-1,3-diol | 4 × 10⁻⁴ | — | — | — | — | — | — |
| | mol | | | | | | |
| 5-Amino-2-methylphenol | — | 4 × 10⁻⁴ | — | — | — | — | — |
| | | mol | | | | | |
| 1H-Indol-6-ol | — | — | 4 × 10⁻⁴ | — | — | — | — |
| | | | mol | | | | |
| 2-Aminopyridin-3-ol | — | — | — | 4 × 10⁻⁴ | — | — | — |
| | | | | mol | | | |
| 3,6-Dimethyl-1H-pyrazolo[5,1-c]- | — | — | — | — | 4 × 10⁻⁴ | — | — |
| [1,2,4]triazole | | | | | mol | | |
| 2-(2,4-Diamino-phenoxy)ethanol | — | — | — | — | — | 4 × 10⁻⁴ | — |
| hydrochloride | | | | | | mol | |
| 3-Amino-2-chloro-6-methylphenol | — | — | — | — | — | — | 4 × 10⁻⁴ |
| hydrochloride | | | | | | | mol |
| Dye support | (2) | (2) | (2) | (2) | (2) | (2) | (2) |
| Demineralized water qs | 100 g | 100 g | 100 g | 100 g | 100 g | 100 g | 100 g |

(2): pH 9.5 Dye Support

| | | |
|---|---|---|
| 96° ethyl alcohol | 20.8 | g |
| Sodium metabisulfite as an aqueous | 0.23 | g A.M. |
| 35% solution | | |
| Pentasodium salt of | 0.48 | g A.M. |
| diethylenetriaminepentaacetic acid | | |
| as an aqueous 40% solution | | |
| C₈-C₁₀ alkyl polyglucoside as an | 3.6 | g A.M. |
| aqueous 60% solution | | |
| Benzyl alcohol | 2.0 | g |
| Polyethylene glycol containing | 3.0 | g |
| 8 units of ethylene oxide | | |
| NH₄Cl | 4.32 | g |
| Aqueous ammonia containing 20% NH₃ | 2.94 | g |

At the time of use, each composition is mixed with an equal weight of 20-volumes aqueous hydrogen peroxide solution (6% by weight). A final pH of 9.5 is obtained.

Each mixture obtained is applied to locks of grey hair containing 90% white hairs. After leaving to act for 30 minutes, the locks are rinsed, washed with a standard shampoo, rinsed again and then dried.

| | Example | | | | | | |
|---|---|---|---|---|---|---|---|
| | 36 | 37 | 38 | 39 | 40 | 41 | 42 |
| Shade | Orange- | Orange- | Orange- | Red | Blue- | Brown | Brown |
| observed | brown | yellow | yellow | | green | | |
| | | | | | grey | | |

### Examples 43 to 56

### Dye Compositions Using 2-[4,7-bis(4-aminophenyl)[1,4,7]triazonan-1-yl]ethanol (8)

### Examples 43 to 49

### Dyeing in Acidic Medium

The following dye compositions are prepared:

| | Example | | | | | | |
|---|---|---|---|---|---|---|---|
| | 43 | 44 | 45 | 46 | 47 | 48 | 49 |
| 2-[4,7-Bis(4-aminophenyl)[1,4,7]tri- | 4 × 10⁻⁴ | 4 × 10⁻⁴ | 4 × 10⁻⁴ | 4 × 10⁻⁴ | 4 × 10⁻⁴ | 4 × 10⁻⁴ | 4 × 10⁻⁴ |
| azonan-1-yl]ethanol | mol | mol | mol | mol | mol | mol | mol |
| Benzene-1,3-diol | 4 × 10⁻⁴ | — | — | — | — | — | — |
| | mol | | | | | | |
| 5-Amino-2-methylphenol | — | 4 × 10⁻⁴ | — | — | — | — | — |
| | | mol | | | | | |
| 1H-Indol-6-ol | — | — | 4 × 10⁻⁴ | — | — | — | — |
| | | | mol | | | | |
| 2-Aminopyridin-3-ol | — | — | — | 4 × 10⁻⁴ | — | — | — |
| | | | | mol | | | |
| 3,6-Dimethyl-1H-pyrazolo[5,1-c]- | — | — | — | — | 4 × 10⁻⁴ | — | — |
| [1,2,4]triazole | | | | | mol | | |
| 2-(2,4-Diamino-phenoxy)ethanol | — | — | — | — | — | 4 × 10⁻⁴ | — |
| hydrochloride | | | | | | mol | |
| 3-Amino-2-chloro-6-methylphenol | — | — | — | — | — | — | 4 × 10⁻⁴ |
| hydrochloride | | | | | | | mol |
| Dye support | (1) | (1) | (1) | (1) | (1) | (1) | (1) |
| Demineralized water qs | 100 g | 100 g | 100 g | 100 g | 100 g | 100 g | 100 g |

(1): pH 7 Dye Support

| | | |
|---|---|---|
| 96° ethyl alcohol | 20.8 | g |
| Sodium metabisulfite as an aqueous | 0.23 | g A.M. |
| 35% solution | | |
| Pentasodium salt of | 0.48 | g A.M. |
| diethylenetriaminepentaacetic acid | | |
| as an aqueous 40% solution | | |
| C₈-C₁₀ alkyl polyglucoside as an | 3.6 | g A.M. |
| aqueous 60% solution | | |
| Benzyl alcohol | 2.0 | g |
| Polyethylene glycol containing | 3.0 | g |
| 8 units of ethylene oxide | | |
| Na₂HPO₄ | 0.28 | g |
| KH₂PO₄ | 0.46 | g |

At the time of use, each composition is mixed with an equal weight of 20-volumes aqueous hydrogen peroxide solution (6% by weight). A final pH of 7 is obtained.

Each mixture obtained is applied to locks of grey hair containing 90% white hairs. After leaving to act for 30 minutes, the locks are rinsed, washed with a standard shampoo, rinsed again and then dried.

| | Example | | | | | | |
|---|---|---|---|---|---|---|---|
| | 43 | 44 | 45 | 46 | 47 | 48 | 49 |
| Shade | Strong | Strong | Strong | Strong | Strong | Strong | Strong |
| observed | red- | red- | red- | grey | red- | blue | violet- |
| | brown | grey | grey | | brown | | grey |

### Examples 50 to 56

### Dyeing in Basic Medium

The following dye compositions are prepared:

| | Example | | | | | | |
|---|---|---|---|---|---|---|---|
| | 50 | 51 | 52 | 53 | 54 | 55 | 56 |
| 2-[4,7-Bis(4-aminophenyl)[1,4,7]tri- | 4 × 10⁻⁴ | 4 × 10⁻⁴ | 4 × 10⁻⁴ | 4 × 10⁻⁴ | 4 × 10⁻⁴ | 4 × 10⁻⁴ | 4 × 10⁻⁴ |
| azonan-1-yl]ethanol | mol | mol | mol | mol | mol | mol | mol |
| Benzene-1,3-diol | 4 × 10⁻⁴ | — | — | — | — | — | — |
| | mol | | | | | | |
| 5-Amino-2-methylphenol | — | 4 × 10⁻⁴ | — | — | — | — | — |
| | | mol | | | | | |
| 1H-Indol-6-ol | — | — | 4 × 10⁻⁴ | — | — | — | — |
| | | | mol | | | | |
| 2-Aminopyridin-3-ol | — | — | — | 4 × 10⁻⁴ | — | — | — |
| | | | | mol | | | |
| 3,6-Dimethyl-1H-pyrazolo[5,1-c]- | — | — | — | — | 4 × 10⁻⁴ | — | — |
| [1,2,4]triazole | | | | | mol | | |
| 2-(2,4-Diamino-phenoxy)ethanol | — | — | — | — | — | 4 × 10⁻⁴ | — |
| hydrochloride | | | | | | mol | |
| 3-Amino-2-chloro-6-methylphenol | — | — | — | — | — | — | 4 × 10⁻⁴ |
| hydrochloride | | | | | | | mol |
| Dye support | (2) | (2) | (2) | (2) | (2) | (2) | (2) |
| Demineralized water qs | 100 g | 100 g | 100 g | 100 g | 100 g | 100 g | 100 g |

(2): pH 9.5 Dye Support

| | | |
|---|---|---|
| 96° ethyl alcohol | 20.8 | g |
| Sodium metabisulfite as an aqueous | 0.23 | g A.M. |
| 35% solution | | |
| Pentasodium salt of | 0.48 | g A.M. |
| diethylenetriaminepentaacetic acid | | |
| as an aqueous 40% solution | | |
| C₈-C₁₀ alkyl polyglucoside as an | 3.6 | g A.M. |
| aqueous 60% solution | | |
| Benzyl alcohol | 2.0 | g |
| Polyethylene glycol containing | 3.0 | g |
| 8 units of ethylene oxide | | |
| NH₄Cl | 4.32 | g |
| Aqueous ammonia containing 20% NH₃ | 2.94 | g |

At the time of use, each composition is mixed with an equal weight of 20-volumes aqueous hydrogen peroxide solution (6% by weight). A final pH of 9.5 is obtained.

Each mixture obtained is applied to locks of grey hair containing 90% white hairs. After leaving to act for 30 minutes, the locks are rinsed, washed with a standard shampoo, rinsed again and then dried.

| | Example | | | | | | |
|---|---|---|---|---|---|---|---|
| | 50 | 51 | 52 | 53 | 54 | 55 | 56 |
| Shade | Orange- | Strong | Strong | Strong | Strong | Blue | Strong |
| observed | red | red | red | red-grey | red-violet | | blue-violet |

### Examples 57 to 70

### Dye Compositions Using 3-{3-[4,7-bis(4-aminophenyl)[1,4,7]triazonan-1-yl]propyl}-1-methyl-3H-imidazol-1-ium chloride (10)

### Examples 57 to 63

### Dyeing in Acidic Medium

The following dye compositions are prepared:

| | Example | | | | | | |
|---|---|---|---|---|---|---|---|
| | 57 | 58 | 59 | 60 | 61 | 62 | 63 |
| 3-{3-[4,7-Bis(4-aminophenyl)[1,4,7]tri- | 4 × 10⁻⁴ | 4 × 10⁻⁴ | 4 × 10⁻⁴ | 4 × 10⁻⁴ | 4 × 10⁻⁴ | 4 × 10⁻⁴ | 4 × 10⁻⁴ |
| azonan-1-yl]-propyl)-1-methyl-3H- | mol | mol | mol | mol | mol | mol | mol |
| imidazol-1-ium chloride | | | | | | | |
| Benzene-1,3-diol | 4 × 10⁻⁴ | — | — | — | — | — | — |
| | mol | | | | | | |
| 5-Amino-2-methylphenol | — | 4 × 10⁻⁴ | — | — | — | — | — |
| | | mol | | | | | |
| 1H-Indol-6-ol | — | — | 4 × 10⁻⁴ | — | — | — | — |
| | | | mol | | | | |
| 2-Aminopyridin-3-ol | — | — | — | 4 × 10⁻⁴ | — | — | — |
| | | | | mol | | | |
| 3,6-Dimethyl-1H-pyrazolo[5,1-c]- | — | — | — | — | 4 × 10⁻⁴ | — | — |
| [1,2,4]triazole | | | | | mol | | |
| 2-(2,4-Diamino-phenoxy)ethanol | — | — | — | — | — | 4 × 10⁻⁴ | — |
| hydrochloride | | | | | | mol | |
| 3-Amino-2-chloro-6-methylphenol | — | — | — | — | — | — | 4 × 10⁻⁴ |
| hydrochloride | | | | | | | mol |
| Dye support | (1) | (1) | (1) | (1) | (1) | (1) | (1) |
| Demineralized water qs | 100 g | 100 g | 100 g | 100 g | 100 g | 100 g | 100 g |

(1): pH 7 Dye Support

| | | |
|---|---|---|
| 96° ethyl alcohol | 20.8 | g |
| Sodium metabisulfite as an aqueous | 0.23 | g A.M. |
| 35% solution | | |
| Pentasodium salt of | 0.48 | g A.M. |
| diethylenetriaminepentaacetic acid | | |
| as an aqueous 40% solution | | |
| C₈-C₁₀ alkyl polyglucoside as an | 3.6 | g A.M. |
| aqueous 60% solution | | |
| Benzyl alcohol | 2.0 | g |
| Polyethylene glycol containing | 3.0 | g |
| 8 units of ethylene oxide | | |
| Na₂HPO₄ | 0.28 | g |
| KH₂PO₄ | 0.46 | g |

At the time of use, each composition is mixed with an equal weight of 20-volumes aqueous hydrogen peroxide solution (6% by weight). A final pH of 7 is obtained.

Each mixture obtained is applied to locks of grey hair containing 90% white hairs. After leaving to act for 30 minutes, the locks are rinsed, washed with a standard shampoo, rinsed again and then dried.

| | Example | | | | | | |
|---|---|---|---|---|---|---|---|
| | 57 | 58 | 59 | 60 | 61 | 62 | 63 |
| Shade | Strong | Strong | Strong | Strong | Orange | Strong | Strong |
| observed | orange- | red-violet | red- | red- | | grey | violet- |
| | brown | grey | brown | brown | | | grey |

### Examples 64 to 70

### Dyeing in Basic Medium

The following dye compositions are prepared:

| | Example | | | | | | |
|---|---|---|---|---|---|---|---|
| | 64 | 65 | 66 | 67 | 68 | 69 | 70 |
| 3-{3-[4,7-Bis(4-aminophenyl)[1,4,7]tri- | 4 × 10⁻⁴ | 4 × 10⁻⁴ | 4 × 10⁻⁴ | 4 × 10⁻⁴ | 4 × 10⁻⁴ | 4 × 10⁻⁴ | 4 × 10⁻⁴ |
| azonan-1-yl]-propyl}-1-methyl-3H- | mol | mol | mol | mol | mol | mol | mol |
| imidazol-1-ium chloride | | | | | | | |
| Benzene-1,3-diol | 4 × 10⁻⁴ | — | — | — | — | — | — |
| | mol | | | | | | |
| 5-Amino-2-methylphenol | — | 4 × 10⁻⁴ | — | — | — | — | — |
| | | mol | | | | | |
| 1H-Indol-6-ol | — | — | 4 × 10⁻⁴ | — | — | — | — |
| | | | mol | | | | |
| 2-Aminopyridin-3-ol | — | — | — | 4 × 10⁻⁴ | — | — | — |
| | | | | mol | | | |
| 3,6-Dimethyl-1H-pyrazolo[5,1-c]- | — | — | — | — | 4 × 10⁻⁴ | — | — |
| [1,2,4]triazole | | | | | mol | | |
| 2-(2,4-Diamino-phenoxy)ethanol | — | — | — | — | — | 4 × 10⁻⁴ | — |
| hydrochloride | | | | | | mol | |
| 3-Amino-2-chloro-6-methylphenol | — | — | — | — | — | — | 4 × 10⁻⁴ |
| hydrochloride | | | | | | | mol |
| Dye support | (2) | (2) | (2) | (2) | (2) | (2) | (2) |
| Demineralized water qs | 100 g | 100 g | 100 g | 100 g | 100 g | 100 g | 100 g |

(2): pH 9.5 Dye Support

| | | |
|---|---|---|
| 96° ethyl alcohol | 20.8 | g |
| Sodium metabisulfite as an aqueous | 0.23 | g A.M. |
| 35% solution | | |
| Pentasodium salt of | 0.48 | g A.M. |
| diethylenetriaminepentaacetic acid | | |
| as an aqueous 40% solution | | |
| C₈-C₁₀ alkyl polyglucoside as an | 3.6 | g A.M. |
| aqueous 60% solution | | |
| Benzyl alcohol | 2.0 | g |
| Polyethylene glycol containing | 3.0 | g |
| 8 units of ethylene oxide | | |
| NH₄Cl | 4.32 | g |
| Aqueous ammonia containing 20% NH₃ | 2.94 | g |

At the time of use, each composition is mixed with an equal weight of 20-volumes aqueous hydrogen peroxide solution (6% by weight). A final pH of 9.5 is obtained.

Each mixture obtained is applied to locks of grey hair containing 90% white hairs. After leaving to act for 30 minutes, the locks are rinsed, washed with a standard shampoo, rinsed again and then dried.

| | Example | | | | | | |
|---|---|---|---|---|---|---|---|
| | 64 | 65 | 66 | 67 | 68 | 69 | 70 |
| Shade | Strong | Strong | Strong | Strong | Strong | Strong | Strong |
| observed | brown | violet | red | red-grey | red-violet | blue | violet |

1. Triazacyclononane derivatives substituted on at least one of the nitrogen atoms with a substituted or unsubstituted 4′-aminophenyl group.

2. Derivatives according to claim 1 of formula (I) below, and the addition salts thereof: 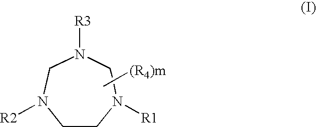
in which:
R₁, R₂ and R₃ represent:
a hydrogen atom;
a saturated or unsaturated alkyl radical, which is unsubstituted or substituted with one or more carboxyl, alkylcarbonyl, alkoxycarbonyl, carbamoyl, mono- or dialkylcarbamoyl, alkylsulfonyl, trialkylammonium or N-alkylimidazolium radicals, or saturated or unsaturated heterocyclic radicals containing 4, 5, 6 or 7 atoms, the hetero atom(s) of which is (are) chosen from nitrogen, oxygen and sulfur;
a radical —CH₂—R′ in which R′ is a saturated or unsaturated alkyl radical substituted with one or more hydroxyl, alkoxy, amino, mono- or dialkylamino, trialkylammonium, alkylcarbonylamino or alkylcarbonyloxy radicals;
a saturated or unsaturated alkylcarbonyl radical, which is unsubstituted or substituted with one or more hydroxyl, alkoxy, amino, mono- or dialkylamino, alkylsulfonyl, carboxyl, alkylcarbonyl, alkoxycarbonyl, carbamoyl, mono- or dialkylcarbamoyl, trialkylammonium or N-alkylimidazolium radicals, or saturated or unsaturated heterocyclic radicals containing 4, 5, 6 or 7 atoms, the hetero atom(s) of which is (are) chosen from nitrogen, oxygen and sulfur;
an alkoxycarbonyl radical;
a mono- or dialkylcarbamoyl radical;
a carbamoyl radical;
a carboxyl radical;
an alkylsulfonyl radical;
an arylsulfonyl radical;
a radical corresponding to formula (II): 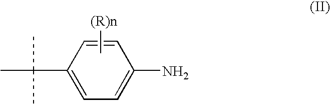
in which:
R represents:
a saturated or unsaturated alkyl radical, which is unsubstituted or substituted with one or more hydroxyl, amino, guanidinyl, trialkylammonium, N-alkylimidazolium, carboxyl or carbamoyl radicals;
a saturated or unsaturated alkoxy radical, which is unsubstituted or substituted with one or more hydroxyl, amino, trialkylammonium or N-alkylimidazolium radicals;
an alkoxyalkyl radical;
a hydroxyalkoxyalkyl radical;
an aminoalkyl radical, the amine possibly being unsubstituted or mono- or disubstituted with an alkyl, acetyl or hydroxyalkyl radical;
n is between 0 and 4, it being understood that when n is greater than 1, then the radicals R may be identical or different;
it being understood that at least one of the radicals R₁, R₂ and R₃ is chosen from the radicals of formula (II);
R₄ represents:
a saturated or unsaturated alkyl radical;
a hydroxyalkyl radical;
an alkoxyalkyl radical;
an alkylcarbonyl radical;
a hydroxyalkoxyalkyl radical;
an aminoalkyl radical, the amine possibly being unsubstituted or mono- or disubstituted with an alkyl, acetyl or hydroxyalkyl radical;
a hydroxyl or aminoalkyl radical;
a carboxyl radical;
a carboxyalkyl radical;
a carbamoyl radical;
a carbamoylalkyl radical;
an alkoxycarbonyl radical;
a mono- or dialkylaminocarbonyl radical;
m is between 0 and 6, it being understood that when m is greater than 1, then the radicals R₄ may be identical or different.

3. Derivatives according to claim 2 of formula (I) in which two of the radicals R₁, R₂ and R₃ are radicals corresponding to formula (II).

4. Derivatives according to claim 2 or 3 of formula (I) in which n is equal to 0 or R is chosen from an alkyl radical; a hydroxyalkyl radical; an aminoalkyl radical; an alkoxy radical; a hydroxyalkoxy radical.

5. Derivatives according to claim 4 of formula (I) in which n is equal to 0 or R is chosen from a methyl radical; a hydroxymethyl radical; a 1,2-dihydroxyethyl radical.

6. Derivatives according to claim 4 or 5 of formula (I) in which n is equal to 0 or 1.

7. Derivatives according to any one of claims 2 to 6 of formula (I) in which the radical(s) R₁, R₂ and/or R₃, which are different from the radicals corresponding to formula (II), are chosen from a hydrogen atom; an alkyl radical; an alkylcarbonyl radical; an alkylsulfonyl radical; a radical —CH₂—R′ in which R′ is an alkyl radical substituted with one or more hydroxyl, alkoxy, amino, mono- or dialkylamino or trialkylammonium radicals; an alkyl radical substituted with one or more trialkylammonium or N-alkylimidazolium radicals; an alkylcarbonyl radical substituted with one or more N-alkylimidazolium radicals; a mono- or dialkylcarbamoyl radical.

8. Derivatives according to claim 7 of formula (I) in which the radical(s) R₁, R₂ and/or R₃, which are different from the radicals corresponding to formula (II), are chosen from a hydrogen atom; a 2-hydroxyethyl radical; a 2,3-dihydroxypropyl radical; a 3-hydroxypropyl radical; a 2-aminoethyl radical; a 2-methoxyethyl radical; a methyl radical; an ethyl radical; an acetyl radical; a methylsulfonyl radical; an N,N-dimethylcarbamoyl radical; a 2-(N,N-dimethylamino)ethyl radical; a (1-methylimidazol-1-ium-3-yl)methylcarbonyl radical; a 3-(trimethylammonium)-2-hydroxypropyl radical; a (1-methylimidazol-1-ium-3-yl)propyl radical; a 2-(trimethylammonium)ethyl radical.

9. Derivatives according to any one of claims 2 to 8 of formula (I) in which m is equal to 0 or R₄ is chosen from an alkyl radical; a hydroxyalkyl radical; a carboxyl radical; a carbamoyl radical.

10. Derivatives according to claim 9 of formula (I) in which m is equal to 0 or 1.

11. Derivatives according to any one of claims 2 and 4 to 10, chosen from 1,4-bis(4-aminophenyl)[1,4,7]triazonane; 1-methyl-4,7-bis(4-aminophenyl)-[1,4,7]triazonane; 2-[4,7-bis(4-aminophenyl)[1,4,7]-triazonan-1-yl]ethanol; 1-ethyl-4,7-bis(4-aminophenyl)-[1,4,7]triazonane; 3-[4,7-bis(4-aminophenyl)[1,4,7]triazonan-1-yl]propane-1,2-diol; 1-acetyl-4,7-bis(4-aminophenyl)[1,4,7]triazonane; 1-(N,N-dimethylcarbamoyl)-4,7-bis(4-aminophenyl)[1,4,7]triazonane; 1-(β-aminoethyl)-4,7-bis(4-aminophenyl)[1,4,7]triazonane; 1-[β-(N,N-dimethylamino)ethyl]-4,7-bis(4-aminophenyl)-[1,4,7]triazonane; 3-{2-[4,7-bis(4-aminophenyl)[1,4,7]-triazonan-1-yl]-2-oxoethyl}-1-methyl-3H-imidazol-1-ium chloride; 1-(γ-hydroxypropyl)-4,7-bis(4-aminophenyl)-[1,4,7]triazonane; 1-(β-methoxyethyl)-4,7-bis(4-aminophenyl)[1,4,7]triazonane; 1,4-bis(4-amino-3-methylphenyl)[1,4,7]triazonane; 1-methyl-4,7-bis(4-amino-3-methylphenyl)[1,4,7]triazonane; 2-[4,7-bis(4-amino-3-methylphenyl)[1,4,7]triazonan-1-yl]ethanol; 1-ethyl-4,7-bis(4-amino-3-methylphenyl)[1,4,7]triazonane; 3-[4,7-bis(4-amino-3-methylphenyl)[1,4,7]triazonan-1-yl]-propane-1,2-diol; 1-acetyl-4,7-bis(4-amino-3-methylphenyl)[1,4,7]triazonane; 1-(N,N-dimethylcarbamoyl)-4,7-bis(4-amino-3-methylphenyl)[1,4,7]triazonane; 1-(β-aminoethyl)-4,7-bis(4-amino-3-methylphenyl)[1,4,7]-triazonane; 1-[β-(N,N-dimethylamino)ethyl]-4,7-bis(4-amino-3-methylphenyl)[1,4,7]triazonane; 3-{2-[4,7-bis(4-amino-3-methylphenyl)[1,4,7]triazonan-1-yl]-2-oxoethyl}-1-methyl-3H-imidazol-1-ium chloride; 3-[4,7-bis(4-amino-3-methylphenyl)[1,4,7]triazonan-1-yl]-propan-1-ol; 1-(β-methoxyethyl)-4,7-bis(4-amino-3-methylphenyl)[1,4,7]triazonane; 1,4,7-tris(4-aminophenyl)[1,4,7]triazonane; 1,4,7-tris(4-amino-3-methylphenyl)[1,4,7]triazonane; 1-(4-aminophenyl)[1,4,7]triazonane; 1,4-dimethyl-7-(4-aminophenyl)[1,4,7]triazonane; 1,4-bis(methanesulfonyl)-7-(4-aminophenyl)[1,4,7]-triazonane; 1,4-diacetyl-7-(4-aminophenyl)[1,4,7]triazonane; 1,4-bis(β-hydroxyethyl)-7-(4-aminophenyl)-[1,4,7]triazonane; 1-(4-aminophenyl)-4,7-bis(γ-trimethylammonium-β-hydroxypropyl)[1,4,7]triazonane; 1-(4-amino-3-methylphenyl)[1,4,7]triazonane; 1,4-dimethyl-7-(4-amino-3-methylphenyl)[1,4,7]triazonane; 1,4-bismethanesulfonyl-7-(4-amino-3-methylphenyl)[1,4,7]-triazonane; 1,4-diacetyl-7-(4-amino-3-methylphenyl)-[1,4,7]triazonane; 1,4-bis(β-hydroxyethyl)-7-(4-amino-3-methylphenyl) [1,4,7]triazonane; 1,4-bis(γ-trimethylammonium-β-hydroxypropyl)-7-(4-amino-3-methylphenyl)-[1,4,7]triazonane chloride; 3-{3-[4,7-bis(4-aminophenyl)[1,4,7]triazonan-1-yl]propyl}-1-methyl-3H-imidazol-1-ium chloride; 3-{3-[4,7-bis(4-amino-3-methylphenyl)[1,4,7]triazonan-1-yl]propyl}-1-methyl-3H-imidazol-1-ium chloride; {2-[4,7-bis(4-aminophenyl)[1,4,7]triazonan-1-yl]ethyl}trimethylammonium chloride; {2-[4,7-bis(4-amino-3-methylphenyl)[1,4,7]-triazonan-1-yl]ethyl}trimethylammonium chloride.

12. Derivatives according to claim 11 of formula (I), chosen from 1-(4-aminophenyl)[1,4,7]-triazonane; 1,4-bis(4-aminophenyl)[1,4,7]triazonane; 1,4,7-tris(4-aminophenyl)[1,4,7]triazonane; 2-[4,7-bis(4-aminophenyl)[1,4,7]triazonan-1-yl]ethanol and 3-{3-[4,7-bis(4-aminophenyl)[1,4,7]triazonan-1-yl]-propyl}-1-methyl-3H-imidazol-1-ium chloride.

13. Use, as oxidation base for the oxidation dyeing of keratin fibres, of a derivative as defined in any one of claims 1 to 12.

14. Composition for dyeing keratin fibres, comprising, in a medium that is suitable for dyeing, at least one derivative as defined in any one of claims 1 to 12 as oxidation base.

15. Composition according to claim 14, in which the amount of derivatives as defined in any one of claims 1 to 12 is between 0.001% and 10% by weight relative to the total weight of the dye composition.

16. Composition according to claim 14 or 15, also comprising a coupler chosen from meta-phenylenediamines, meta-aminophenols, meta-diphenols, naphthalene-based couplers and heterocyclic couplers, and the addition salts thereof.

17. Composition according to claim 16, in which the amount of each of the couplers is between 0.001% and 10% by weight relative to the total weight of the dye composition.

18. Composition according to any one of claims 14 to 17, comprising an additional base chosen from para-phenylenediamines other than the derivatives as defined in any one of claims 1 to 12, bis(phenyl)-alkylenediamines, para-aminophenols, bis-para-aminophenols, ortho-aminophenols, ortho-phenylenediamines and heterocyclic bases, and the addition salts thereof.

19. Composition according to claim 18, in which the amount of each of the additional oxidation bases is between 0.001% and 10% by weight relative to the total weight of the dye composition.

20. Process for dyeing keratin fibres, characterized in that a composition as defined in any one of claims 14 to 19 is applied to the keratin fibres in the presence of an oxidizing agent for a time that is sufficient to develop the desired coloration.

21. Process according to claim 20, in which the oxidizing agent is chosen from hydrogen peroxide, urea peroxide, alkali metal bromates, persalts, peracids and oxidase enzymes.

22. Multi-compartment device in which a first compartment contains a dye composition as defined in any one of claims 14 to 19 and a second compartment contains an oxidizing agent.

23. Intermediate triazacyclononane derivatives substituted on at least one of the nitrogen atoms with a substituted or unsubstituted 4′-nitrophenyl group.

24. Derivatives according to claim 23 of formula (IV) below, and the addition salts thereof: 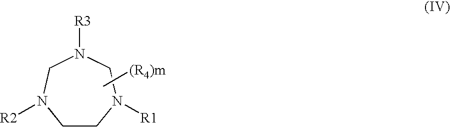
in which:
R₁, R₂ and R₃ represent:
a hydrogen atom;
a saturated or unsaturated alkyl radical, which is unsubstituted or substituted with one or more carboxyl, alkylcarbonyl, alkoxycarbonyl, carbamoyl, mono- or dialkylcarbamoyl, alkylsulfonyl, trialkylammonium or N-alkylimidazolium radicals, or saturated or unsaturated heterocyclic radicals containing 4, 5, 6 or 7 atoms, the hetero atom(s) of which is (are) chosen from nitrogen, oxygen and sulfur;
a radical —CH₂—R′ in which R′ is a saturated or unsaturated alkyl radical substituted with one or more hydroxyl, alkoxy, amino, mono- or dialkylamino, trialkylammonium, alkylcarbonylamino or alkylcarbonyloxy radicals;
a saturated or unsaturated alkylcarbonyl radical, which is unsubstituted or substituted with one or more hydroxyl, alkoxy, amino, mono- or dialkylamino, alkylsulfonyl, carboxyl, alkylcarbonyl, alkoxycarbonyl, carbamoyl, mono- or dialkylcarbamoyl, trialkylammonium or N-alkylimidazolium radicals, or saturated or unsaturated heterocyclic radicals containing 4, 5, 6 or 7 atoms, the hetero atom(s) of which is (are) chosen from nitrogen, oxygen and sulfur;
an alkoxycarbonyl radical;
a mono- or dialkylcarbamoyl radical;
a carbamoyl radical;
a carboxyl radical;
an alkylsulfonyl radical;
an arylsulfonyl radical;
a radical corresponding to formula (V): 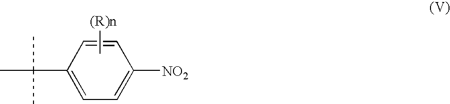
in which:
R represents:
a saturated or unsaturated alkyl radical, which is unsubstituted or substituted with one or more hydroxyl, amino, guanidinyl, trialkylammonium, N-alkylimidazolium, carboxyl or carbamoyl radicals;
a saturated or unsaturated alkoxy radical, which is unsubstituted or substituted with one or more hydroxyl, amino, trialkylammonium or N-alkylimidazolium radicals;
an alkoxyalkyl radical;
a hydroxyalkoxyalkyl radical;
an aminoalkyl radical, the amine possibly being unsubstituted or mono- or disubstituted with an alkyl, acetyl or hydroxyalkyl radical;
n is between 0 and 4, it being understood that when n is greater than 1, then the radicals R may be identical or different;
it being understood that at least one of the radicals R₁, R₂ and R₃ is chosen from the radicals of formula (II);
R₄ represents:
a saturated or unsaturated alkyl radical;
a hydroxyalkyl radical;
an alkoxyalkyl radical;
an alkylcarbonyl radical;
a hydroxyalkoxyalkyl radical;
an aminoalkyl radical, the amine possibly being unsubstituted or mono- or disubstituted with an alkyl, acetyl or hydroxyalkyl radical;
a hydroxyl or aminoalkyl radical;
a carboxyl radical;
a carboxyalkyl radical;
a carbamoyl radical;
a carbamoylalkyl radical;
an alkoxycarbonyl radical;
a mono- or dialkylaminocarbonyl radical;
m is between 0 and 6, it being understood that when m is greater than 1, then the radicals R₄ may be identical or different.
